(19) United States
(12) Patent Application Publication
(10) Pub. No.: US 2023/0149694 A1
(43) Pub. Date: May 18, 2023

(54) MECHANICAL CIRCULATORY SUPPORT DEVICE

(71) Applicant: Medtronic, Inc., MINNEAPOLIS, MN (US)

(72) Inventors: Mustafa Ertan Taskin, Durham, NC (US); Fernando Casas, Miami Lakes, FL (US)

(21) Appl. No.: 17/454,954

(22) Filed: Nov. 15, 2021

(51) Int. Cl. A61M 60/148; A61M 60/419; A61M 60/216; A61M 60/422; A61M 60/824; A61M 60/82

(52) U.S. Cl. CPC A61M 60/148; A61M 60/419; A61M 60/216; A61M 60/422; A61M 60/824; A61M 60/82; A61M 2205/3331

(57) ABSTRACT

In some examples, a medical system includes a pump is configured to provide a pulsating blood flow. The pump may provide the pulsating flow to assist the pumping action of a heart. An impeller is configured to impart energy to the blood flow when the impeller rotates around an eye axis extending through an impeller eye defined by the impeller. The pump includes a magnetic bearing configured such that, as the impeller rotates around the eye axis, the eye axis translates around a post axis defined by a post mechanically supported by a pump housing. The medical system may include a controller configured to control a bearing magnetic field and/or a stator magnetic field to control a pressure of the pulsating flow and/or a speed of the pump.

### TECHNICAL FIELD

This disclosure is related to a medical mechanical circulatory support device.

### BACKGROUND

A mechanical circulatory support device is configured to aid a heart of a patient with pumping blood to the body. An example mechanical circulatory support device is a ventricular assist device, which is configured to assist the pumping action of a heart and may include an inlet, an outlet, and a blood pump arranged to pump blood flow from the inlet to the outlet. The inlet may be fluidically connected to a chamber of the heart of a patient and the outlet may be fluidically connected to an artery, such as an aorta or a pulmonary artery. A blood pump of the ventricular assist device is configured to drive blood from the inlet towards the outlet and thus assists blood flow from the chamber of the heart into the artery. In patients with some degree of heart failure, the ventricular assist device may augment the pumping of the heart to provide blood flow at a sufficient rate to meet the demands of the body. The ventricular assist device may serve as, for example, a bridge to recovery, a bridge to transplantation, or destination therapy for the patient.

### SUMMARY

This disclosure describes a mechanical circulatory support device configured to provide a pulsating blood flow to aid a heart of a patient in pumping blood to the body. A medical pump of the mechanical circulatory support device includes an impeller configured to impart energy to the blood flow when the impeller rotates around an eye axis of an impeller eye defined by the impeller. The blood pump includes a magnetic bearing configured such that, as the impeller rotates around the eye axis, the eye axis translates around (e.g., orbits) around a post axis defined by a post mechanically supported by the pump housing. The translation of the eye axis around the post axis as the impeller rotates transfers energy to the blood flow and imparts energy to the blood flow, which enables the mechanical circulatory support device to generate a pulsating blood flow. The pulsing blood flow can be generated, for example, without changing a speed of rotation of the impeller.

In an example, a mechanical circulatory support device comprises: a housing including a fluid inlet and a fluid outlet, wherein the housing defines a volume fluidically coupling the fluid inlet and the fluid outlet; a post mechanically supported by the housing within the volume, wherein the post defines a post axis, and wherein the post mechanically supports an inner ring; and an impeller mechanically supporting an outer ring, wherein the impeller defines an eye surrounding the post and an eye axis extending through the eye, wherein the inner ring is configured to magnetically interact with the outer ring to establish a radial clearance between the inner ring and the outer ring, and wherein the inner ring is configured to magnetically interact with the outer ring to cause the eye axis to translate around the post axis as the impeller rotates around the eye axis when the impeller imparts energy to a fluid flowing from the fluid inlet to the fluid outlet.

In an example, a heart pump comprises: a housing including a fluid inlet and a fluid outlet, wherein the housing defines a volume fluidically coupling the fluid inlet and the fluid outlet; a stator configured to generate a stator magnetic field; a post mechanically supported by the housing within the volume, wherein the post mechanically supports an inner ring; and an impeller configured to impart energy to a fluid flowing from the fluid inlet to the fluid outlet, wherein the impeller defines an outer radial clearance between an outer perimeter of the impeller and the housing, and wherein the inner ring is configured to magnetically interact with an outer ring mechanically supported by the impeller to cause the outer radial clearance to vary.

In an example, a method comprises: controlling, by control circuitry, an impeller of a medical pump to rotate within a housing, the medical pump comprising: a housing including a fluid inlet and a fluid outlet, wherein the housing defines a volume fluidically coupling the fluid inlet and the fluid outlet; a post mechanically supported by the housing within the volume, wherein the post defines a post axis, and wherein the post mechanically supports an inner ring; and the impeller mechanically supporting an outer ring, wherein the impeller defines an eye surrounding the post and an eye axis extending through the eye, wherein the inner ring is configured to magnetically interact with the outer ring to establish a radial clearance between the inner ring and the outer ring, and wherein the inner ring is configured to magnetically interact with the outer ring to cause the eye axis to translate around the post axis as the impeller rotates around the eye axis when the impeller imparts energy to a fluid flowing from the fluid inlet to the fluid outlet; and modifying, by the control circuitry, a magnetic field causing the magnetic interaction between the inner ring and the outer ring.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example medical system configured to assist the pumping action of a heart, the medical system including a mechanical circulatory support device.

FIG. 2A is a perspective illustration of an example mechanical circulatory support device in a first configuration.

FIG. 2B is a perspective illustration of the mechanical circulatory support device of FIG. 2A in a second configuration.

FIG. 3 illustrates an example pressure waveform of a pulsating flow.

FIG. 4A is a schematic illustration of an example impeller of an example mechanical circulatory support device in a first rotational position within a pump housing.

FIG. 4B is a schematic illustration of the impeller of FIG. 4A in a second rotational position within the pump housing of FIG. 4A.

FIG. 4C is a schematic illustration of the impeller of FIG. 4A-4B in a third rotational position within the pump housing of FIG. 4A.

FIG. 4D is a schematic illustration of the impeller of FIG. 4A-4C in a fourth rotational position within the pump housing of FIG. 4A-4C.

FIG. 5 is a conceptual diagram illustrating a cross-section of an example pump, the cross-section being taken through a post axis defined by the pump.

FIG. 6 is a schematic block diagram of the example medical system.

FIG. 7 is a flow diagram of an example technique of using a mechanical circulatory support device described herein.

### DETAILED DESCRIPTION

This disclosure describes example medical systems configured to augment the pumping of a heart of a patient, e.g., to provide blood flow to meet the demands of the body of a patient during, for example, treatment of heart failure. The medical system includes a mechanical circulatory support device (“MCS device”) including a medical pump (also referred to herein more generally as a “pump”) configured to pump fluid (e.g., blood), which aids a heart of the patient in pumping blood to the body. In some examples, the MCS device is a ventricular assist device (“VAD”). In some examples, the MCS device may be implanted in the body of the patient and powered by an electrical power source inside or outside the body.

The pump is configured to drive blood flow from an inlet of the MCS device to an outlet of the MCS device. Blood may be introduced into the inlet from any suitable location in a body of a patient and blood may be introduced into any suitable part of the body via the outlet. For example, the medical system may include an inflow line (e.g., an inflow cannula) connected to a chamber (e.g., a ventricle) of the heart of the patient and to an inlet of the MCS device, and an outflow line (e.g., an outflow cannula) connected to an artery, such as an aorta or a pulmonary artery, and to an outlet of the MCS device. The pump is configured to drive blood from the inflow line towards the outflow line and thus assist blood flow from the chamber of the heart into the artery.

The pump is configured to provide a pulsating flow when the pump drives blood from the inflow line towards the outflow line. The pulsating flow may define a substantially continuous pressure waveform which substantially oscillates between a maximum pressure and a minimum pressure. The pump may be configured to produce the pulsating flow by at least varying a radial clearance between an impeller and a pump housing substantially surrounding the impeller as the impeller rotates and imparts energy to the blood flow. The varying radial clearance may at least in part cause the pump to produce the pulsating flow. Hence, in some examples, the pump is configured to produce the pulsating flow when the impeller rotates at a substantially constant rotational speed (e.g., constant or nearly constant to the extent permitted by device tolerances). In some examples, in addition to or instead of varying the radial clearance, the pump varies the rotational speed of the impeller to produce the pulsating flow.

The pulsating flow may help assist and/or substantially mimic the pulsatile flow naturally produced by the heart. The pulsating flow may, for example, complement the ejection of blood by the heart in systole, which may help relieve the work of the heart. As the heart relaxes in diastole, the pulsating flow may assist in increasing blood pressure when the pulsating flow provides blood to the aorta. The improved perfusion and reduced work performed by the heart may improve the performance of the heart, improving circulation for the entire body of the patient. In addition, in some cases, the pulsating flow provided by the MCS device may help disrupt flow in a ventricle of the patient and/or help wash the pump, which can help minimize thrombus ingestion by the pump.

The pump includes an impeller configured to generate the pulsating fluid flow when the impeller rotates to impart energy to a blood flow in the MCS device. In addition to or instead of achieving a pulsating flow by varying a speed of the pump (e.g., a speed of rotation of the impeller), the MCS devices described herein are configured to achieving a pulsating fluid flow by at least modifying the path the impeller takes as it rotates within a pump housing of the pump. The impeller is magnetically and/or hydrodynamically suspended within a pump housing of the pump, such that the impeller maintains a radial and axial clearance from the pump housing as the impeller rotates. Rather than rotating concentrically around a center post and maintaining a constant gap between the impeller and the pump housing, the pumps described herein are configured such that the radial clearance from the pump housing varies as the impeller rotates. That is, as the magnetically suspended impeller rotates, an axis of rotation of the impeller may vary relative to a post axis defined by the center post, such that the radial clearances between an outer perimeter of the impeller and the pump housing vary. In examples, the axis of rotation of the magnetically suspended impeller translates around (e.g., substantially orbits) the post axis as the magnetically suspended impeller rotates (e.g., as a fixed body) around the axis of rotation. As a result, the forces imparted by the impeller on fluid flowing through the pump housing are non-homogenous, and impeller motion may cause variations in the pressure of the fluid flowing through the pump housing.

Varying a speed of rotation of the impeller (also referred to herein as pump speed) may be useful for achieving a pulsatile flow through the pump, but changing the speed of rotation of the impeller relatively quickly can be relatively difficult to achieve due to limitations of the hardware. In addition, varying the pump speed to achieve pulsatile blood flow may consume more power than modifying the path the impeller takes as it rotates within a pump housing of the pump to achieve pulsatile flow by a MCS device.

The pumps described herein are configured such that, as the impeller rotates around an eye axis extending through an impeller eye defined by the impeller, the eye axis translates around (e.g., orbits) around a post axis defined by a post mechanically supported by the pump housing. The post can be, for example, fixed or integral with the pump housing and stationary relative to the pump housing during operation of the pump. In examples, the impeller eye surrounds the post when the impeller rotates within the pump housing. The translation of the eye axis around the post axis as the impeller transfers energy to the blood flow (e.g., increases a velocity of the flow) may cause the pump to generate a pulsating blood flow. In examples, the pumps described herein cause an increase in pressure (e.g., a static pressure) of the blood flow in regions where the translation of the eye axis around the post axis causes a decrease in the radial clearance between the impeller and the pump housing. The pumps described herein may cause an decrease in pressure (e.g., a static pressure) of the blood flow in regions where the translation of the eye axis around the post axis causes an increase in the radial clearance between the impeller and the pump housing. The varying pressure resulting from the varying radial clearance may generate the pulsating flow. Thus, the pumps described herein are configured to provide a pulsating (e.g., pulsatile) blood flow, even while the pump is operating at a substantially constant pump speed (e.g., constant or nearly constant to the extent permitted by manufacturing tolerances).

The pump includes a magnetic bearing (e.g., part of the impeller suspension system) configured to enable the eye axis to translate around the post axis as the impeller rotates. In some examples, the magnetic bearing includes an inner ring mechanically supported by the post and an outer ring mechanically supported by the impeller. The magnetic bearing is configured to generate a magnetic field (“bearing magnetic field”) to cause the inner ring to magnetically interact with the outer ring. In examples, the inner ring and the outer ring define the bearing magnetic field. The magnetic interaction may cause magnetic forces (e.g., attractive or repulsive forces) between inner ring and the outer ring. The inner ring may be configured to transmit some portion of the magnetic forces to the post and the outer ring may be configured to transmit some portion of the magnetic forces to the impeller, such that the magnetic forces establish an inner radial clearance between the post and the impeller (e.g., a radial clearance substantially perpendicular to the eye axis and/or post axis). In some examples, the magnetic bearing is configured to generate the bearing magnetic field such that the magnetic forces cause the inner radial clearance between the impeller and the post axis to vary around a perimeter defined by the post (e.g., a perimeter in a plane substantially perpendicular to the post axis). The magnetic forces may cause the inner radial clearance to vary such that, when the magnetic bearing establishes the inner radial clearance, the eye axis substantially offsets from the post axis, such that the eye axis translates around post axis when the impeller rotates to impart energy to a blood flow through the MCS device.

For example, in some examples, the magnetic bearing is configured such that a first magnetic force causes an inner radial clearance between the post and the impeller in a first radial direction (e.g., a direction substantially perpendicular to the post axis). The magnetic bearing may further be configured such that a second magnetic force causes an inner radial clearance between the post and the impeller in a second direction substantially opposite the first direction. The second magnetic force may be less than the first magnetic force. For example, the second magnetic force may be less than about 90% of the first magnetic force, such as less than about 70% or 50% of the first magnetic force, or less than some other percentage of the first magnetic force sufficient to cause the offset between the eye axis and the post axis. The differing magnetic forces may cause the eye axis to translate around post axis when the impeller rotates to impart energy to the blood flow, such that pump provides a pulsating blood flow.

The pump is configured such that the magnetic forces between the impeller and the post vary around the perimeter defined by the post and/or the inner ring. For example, in some examples, the inner ring and/or the outer ring includes a permanent magnet configured to cause the magnetic forces between the impeller and the post to vary around the perimeter defined by the post. In some cases, the permanent magnet defines a structural feature (e.g., a notch in the permanent magnet) configured to cause the magnetic forces to vary. In addition or instead, in some examples, the inner ring and/or the outer ring includes an electromagnet configured to cause the magnetic forces to vary. In some examples, the electromagnet includes a winding configured to generate an electromagnetic field to generate the magnetic forces, and the winding is configured to cause the magnetic forces to vary around the perimeter defined by the post and/or the inner ring.

The pump housing defines a fluid inlet and a fluid outlet. The impeller is configured to rotate within the pump housing to impart energy to the blood between the fluid inlet and the fluid outlet. The impeller may be configured to impart energy to the blood flow by at least accelerating the blood flow toward an outer perimeter (“impeller outer perimeter”) defined by the impeller as the impeller rotates. The impeller outer perimeter may be defined by, for example, one or more vane tips of the impeller. The pump housing may define a volume (e.g., a volute) fluidically coupling the fluid inlet and the fluid outlet and configured to receive the blood flow accelerated by the impeller. In some examples, the fluid inlet is fluidically coupled to the inflow line and the fluid outlet is fluidically coupled to the outflow line, such that rotation of the impeller drives blood from the inflow line towards the outflow line. The eye axis about which the impeller is configured to rotate may be extend through an impeller eye defined by the impeller. The pump may be configured such that the eye axis defines the axis of rotation for the impeller when the impeller rotates to accelerate the blood flow.

In examples, the pump is configured to generate a pulsating flow wherein a pressure of the flow within the pump housing (e.g., at the fluid outlet) cyclically varies over a pressure range defined by a maximum pressure and a minimum pressure as the impeller rotates within the housing. In examples, during one full translation of the eye axis around the post axis, the pressure of the flow within the pump housing varies between the maximum and minimum pressures. The pulsating flow may define a substantially continuous pressure waveform which substantially oscillates between the maximum pressure and the minimum pressure. In examples, the pressure waveform defines a wave period.

In some examples, the medical system is configured to control the maximum pressure, the minimum pressure, and/or the wave period by at least controlling the motion of the impeller. For example, the maximum and/or minimum pressure may be a function of a radial clearance between the impeller and the pump housing as the impeller rotates around the eye axis. Control circuitry of the medical system (e.g., of the MCS device or another device) may be configured to control the wave period by at least controlling a rotational speed of the impeller around the eye axis. In some examples, the control circuitry is configured to receive a signal indicative of a physiological parameter of a patient and cause the pump to alter the pulsating flow based on the physiological parameter. The signal may be received directly or indirectly from a physiological sensor. The physiological parameter may be, for example, a cardiac signal such as an electrocardiogram (ECG), an electrogram (EGM), a signal indicative of an activity level of the patient (e.g., a signal generated by an accelerometer), a mechanical wave of the heart, a signal indicative of a respiratory rate, or another physiological parameter. Hence, the medical system may be configured to alter the pulsating flow provided by the MCS device to augment the pumping of a heart of a patient based on a sensed physiological condition of the patient.

During operation of the pump, an outer radial clearance between the pump housing and the impeller outer perimeter varies as the impeller rotates to generate the pulsating flow. The pump may be configured such that the translation of the eye axis around the post axis causes the outer radial clearance to vary. For example, at an initial point in time, with the impeller rotating around the eye axis and the eye axis translating (e.g., orbiting) round the post axis, the pump may define a first outer radial clearance between the impeller and the pump housing in a first radial direction and a second outer radial clearance between the impeller and the pump housing in a second radial direction opposite the first radial direction. The first outer radial clearance may be less than the second outer radial clearance. At a subsequent point in time, with the impeller having rotated by some degree around the eye axis and the eye axis having translated by some degree round the post axis, the impeller may have altered its orientation relative to the pump housing such that the first outer radial clearance is greater than the second radial outer displacement. The radial clearance can be measured in a direction orthogonal to the post axis.

In some examples, the pump is configured such that the translation of the eye axis around the post axis cause the first outer radial clearance to cyclically vary from a minimum first radial clearance to a maximum first radial clearance as the eye axis translates around the post axis. In examples, as the eye axis translates around the post axis, the first radial clearance defines a substantially continuous first displacement waveform which oscillates between the maximum first radial clearance and the minimum first radial clearance. The first displacement waveform defines a first displacement wave period. In examples, the pump is configured such that the first displacement wave period is substantially equal to the wave period of the pressure waveform defined by the pulsating flow. In similar manner, the pump may be configured such that the translation of the eye axis around the post axis cause the second outer radial clearance to cyclically vary from a minimum second radial clearance to a maximum second radial clearance as the eye axis translates around the post axis. The second radial clearance defines a substantially continuous second displacement waveform which oscillates between the maximum second radial clearance and the minimum second radial clearance as the eye axis translates around the post axis. The second displacement waveform defines a second displacement wave period. The pump may be configured such that the second displacement wave period is substantially equal to the wave period of the pressure waveform defined by the pulsating flow and/or the first displacement wave period.

In some examples, the pump is configured such that the pump housing and the varying magnetic forces between the inner and outer ring cause a side thrust on the impeller when the impeller rotates to impart energy (e.g., a velocity) to the blood flow and enable the pump to generate a pulsating (e.g., pulsatile) fluid flow. The side thrust causes the eye axis to translate around (e.g., orbit) the post axis. The side thrust may, for example, cause the first outer radial clearance and/or the second outer radial clearance to vary from its respective maximum to its respective minimum. In examples, the pump housing and the bearing magnetic field are configured to cause a side thrust on the impeller in a direction from the first outer radial clearance toward the second outer radial clearance when the first outer radial clearance is less than the second outer radial clearance. The pump housing and the bearing magnetic field may be configured to cause a side thrust on the impeller in a direction from the second outer radial clearance toward the first outer radial clearance when the second outer radial clearance is less than the first outer radial clearance. The side thrust causing the eye axis to translate around the post axis as the impeller imparts energy to the blood flow may cause the impeller to generate the pulsating blood flow, such that a pressure of the blood flow at the fluid outlet of the housing cyclically varies substantially from a maximum pressure to a minimum pressure (and vice-versa) as the impeller rotates.

In some examples, for a given MCS device, the bearing magnetic field is modifiable, e.g., to achieve different amounts of fluid pulsations, which may have different physiological effects on the patient. For example, a medical system including the MCS device can include control circuitry configured to control the bearing magnetic field based on user input, based on one or more sensed physiological parameters, or based on other factors or combination thereof. In some examples, the control circuitry is configured to vary the bearing magnetic field in a radial direction (e.g., substantially perpendicular to the eye axis and/or post axis) to cause the magnetic field strength of the magnetic field to vary around the perimeter defined around the post axis. The control circuitry may be configured to alter an operation of the pump (e.g., by at least altering the bearing magnetic field) to control a pressure range exhibited by the pulsations as the impeller generates the pulsating flow. For example, in some examples, the control circuitry is configured to alter the bearing magnetic field to increase a difference between the minimum first radial clearance and the maximum first radial clearance as the eye axis translates around the post axis to, for example, increase the pressure range exhibited by the pulsations. As another example, the control circuitry can be configured to alter the bearing magnetic field to decrease a difference between the minimum first radial clearance and the maximum first radial clearance as the eye axis translates around the post axis to, for example, decrease the pressure range exhibited by the pulsations. Hence, the medical system may be configured to alter the pressure range exhibited by the pulsations depending on the needs of the patient.

In some examples, the medical system includes a programming computing device or the like that is configured to communicate with the control circuitry. A user can interact with the programming computing device to provide user input that causes the control circuitry to modify the pressure range, e.g., by directly specifying different bearing magnetic fields, different pressure ranges, or the like. The control circuitry of the MCS device is configured to, in response to receiving the user input, modify the pressure range, such as by modifying the bearing magnetic field or taking another action described herein that has an impact on the pressure range. In this way, once implanted in the patient, the operation of the MCS device can be modified to accommodate the needs of the patient and provide better therapeutic outcomes, e.g., as the patient condition changes over time without requiring explanation of the device from the patient.

The MCS device is configured to cause the impeller to rotate around the eye axis as the eye axis translates around the post axis. In some examples, the pump defines a stator configured to cause the impeller to rotate around the eye axis. The stator can be, for example, mechanically supported by the pump housing. In examples, the stator and the impeller are configured to magnetically interact to cause the impeller to rotate around the eye axis. The stator and the impeller may define a motor (e.g., a brushless DC motor) configured to exert a torque on the impeller causing the impeller to rotate around the eye axis. In examples, the stator and/or the impeller are configured to define a magnetic field of the motor (“motor magnetic field”) to cause the impeller to rotate around the eye axis. For example, the stator may be configured to generate a rotating motor magnetic field (e.g., rotating substantially around the post axis) to cause the impeller to rotate around the eye axis. The control circuitry of the pump may be configured to control a rotational speed of the impeller by at least, for example, controlling a rotational speed of the motor magnetic field and/or the torque exerted on the impeller (e.g., by controlling a strength of the motor magnetic field). In examples, the pump is configured such that the motor magnetic field and/or hydrodynamic forces from the blood flow cause an axial displacement between the pump housing and the impeller, such that the impeller may rotate within the pump housing to cause the pulsating flow without contacting the pump housing.

In some examples, the control circuitry of the medical system is configured to control an operation of the pump based on a sensed physiological parameter of the patient. For example, the medical system may include a monitor (e.g., an implantable, wearable, or other monitor) including sensing circuitry configured to generate an output (e.g., an electrical signal) indicative of a physiological parameter of a patient (e.g., a cardiac signal such as an ECG, ECM, a signal indicative of an activity level of the patient, a signal indicative of a respiratory rate, a mechanical wave of the heart, or another physiological parameter). The monitor may be configured to communicate a signal indicative of the physiological parameter to the control circuitry. The control circuitry may receive the signal and alter an operation of the pump (e.g., alter the bearing magnetic field and/or the motor magnetic field) based on the received signal. For example, the control circuitry may alter the bearing magnetic field and/or motor magnetic field to alter the radial clearances exhibited between the impeller and the pump housing to, for example, increase or decrease a pressure range of the pulsating flow based on the indicative signal, or for another reason. As another example, the control circuitry may alter the bearing magnetic field and/or motor magnetic field (e.g., a rotation of the motor magnetic field) to alter a rotational speed of the impeller to, for example, increase or decrease a wave period of the pulsating flow based on the indicative signal, alter a flow rate of the blood flow through the pump, or for other reasons.

In some examples, instead of or in addition to controlling operation of the pump based on one or more sensed physiological parameters, the medical system is configured to monitor a blood flow through the pump and alter the operation of the pump based on the monitored blood flow. For example, the medical system may include a pump thrombosis detection system including circuitry configured to detect a pump thrombosis within the pump. The pump thrombosis detection system may be configured to communicate a thrombosis signal indicative of the pump thrombosis to the control circuitry. The control circuitry may be configured to alter an operation of the pump (e.g., alter the bearing magnetic field and/or the motor magnetic field) based on the thrombosis signal. For example, the control circuitry may be configured to alter the bearing magnetic field and/or the motor magnetic field to generate a mechanical vibration in the impeller to try to clear the pump thrombosis.

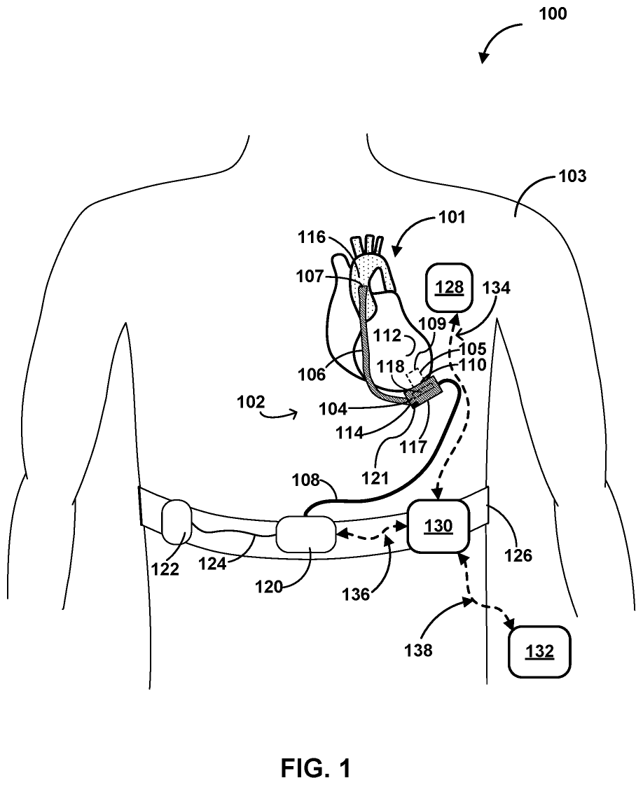

FIG. 1 is a conceptual and schematic diagram illustrating an example medical system 100 configured to assist the pumping action of a heart 101 of a patient 103 using a mechanical circulatory support device 102 (“MCS device 102”). MCS device 102 includes a pump 104. Medical system 100 includes an inflow line 105, an outflow line 106, and a driveline 108. Inflow line 105 and/or outflow line 106 may be, for example, cannulas or other structures configured to define a fluid pathway for blood. A first end of inflow line 105 is fluidically coupled to a fluid inlet 110 of pump 104 either directly or indirectly. A second end of inflow line 105 is fluidically coupled to heart 101, e.g., grafted to heart 101, such as the left ventricle 112 of heart 101. The second end of inflow line 105 may define an MCS inlet 109 fluidically coupled to fluid inlet 110. In some examples, the second end of inflow line 105 includes a ventricular connector (not shown). A first end of outflow line 106 is fluidically coupled to fluid outlet 114 of pump 104 either directly or indirectly. A second end of outflow line 106 may be grafted or otherwise fluidically coupled to an artery of patient 103, e.g., aorta 116. The second end of outflow line 106 may define an MCS outlet 107 fluidically coupled to fluid outlet 114. Driveline 108 is configured to provide electrical and/or mechanical power to pump 104.

Pump 104 is configured to draw blood from a chamber of heart 101 and pump the blood to other portions of the body of patient 103. In examples, pump 104 includes a pump housing 117 and an impeller 118 configured to rotate within pump housing 117 to draw blood from the chamber of heart 101 and pump the blood to other portions of the body of patient 103. Impeller 118 is configured impart energy to a blood flow flowing from inflow line 105 to outflow line 106 when impeller 118 rotates within pump housing 117. In examples, pump 104 is configured to cause impeller 118 to generate a pulsating flow as the blood flow flows from inflow line 105 to outflow line 106. The pulsating flow may define a substantially continuous pressure waveform (e.g., at fluid outlet 114) which oscillates between a maximum pressure and a minimum pressure.

Driveline 108 is configured to provide electrical and/or mechanical power to pump 104 to, for example, cause impeller 118 to impart energy to the blood flow. In the example shown in FIG. 1, medical system 100 includes control circuitry 120 configured to supply and/or control the power supplied by driveline 108. In some examples, control circuitry 120 or other control circuitry of system 100 is configured to control a bearing magnetic field and/or a stator magnetic field generated within pump 104. Thus, while control circuitry 120 is primarily referred to herein for ease of description, in other examples, system 100 can include other control circuitry configured to perform some or all of the functions described with respect to control circuitry 120.

Control circuitry 120 is configured to communicate with pump 104 (e.g., using driveline 108 and/or another communication link) to alter an operation of pump 104. For example, control circuitry 120 may be configured to alter the operation of pump 104 to control a pressure range exhibited by the pulsations of the pulsating flow as impeller 118 imparts energy to the blood flow flowing from inflow line 105 to outflow line 106. As another example, in some examples, control circuitry 120 is configured to alter the operation of pump 104 (e.g., a rotational speed of impeller 118) to control a wave period of the pulsating flow as impeller 118 imparts energy to the blood flow flowing from inflow line 105 to outflow line 106. Control circuitry 120 may be configured to alter the operation of the bearing magnetic field and/or the stator magnetic field within pump 104 to alter the operation of pump 104.

In examples, control circuitry 120 is configured to monitor MCS device 102 (e.g., pump 104) and/or a blood flow produced by MCS device to evaluate the pulsating flow and, in some examples, control pump 104 accordingly. For example, control circuitry 120 may be configured to monitor MCS device 102 to evaluate when pump 104 is operating in a condition causing the eye axis defined by impeller 118 to translate around the post axis as impeller 118 rotates around the eye axis (e.g., when the eye axis is an axis of rotation of impeller 118). In addition, or alternatively, control circuitry 120 may be configured to monitor MCS device 102 to evaluate when pump 104 is operating in a condition causing the radial clearance between impeller 118 and pump housing 117 to vary as impeller 118 rotates around the eye axis. Control circuitry 120 may be configured to alter and/or establish an operating condition of MCS device 102 (e.g., pump 104) to cause MCS device to produce the pulsating flow. Control circuitry 120 may be configured to evaluate a waveform (e.g., a continuous, discontinuous, and/or discrete waveform) generated by MCS device 102 and indicative of a pulsating flow to evaluate a sufficiency of the pulsating flow. Control circuitry 120 may be configured to alter and/or establish an operating condition of MCS device 102 based on the evaluation of the waveform, e.g., to modify the waveform.

In examples, MCS device 102 includes a sensor 121 including sensing circuitry configured to monitor the pulsating flow generated by MCS device 102 and/or a characteristic of MCS device 102 (e.g., pump 104) indicating a pulsating flow. Sensor 121 is configured to generate a flow signal indicative of the pulsating flow and/or the characteristic of MCS device 102 indicating the pulsating flow. For example, sensor 121 may be configured to measure a flow rate and/or pressure of the blood flow in a particular portion of MCS device 102 (e.g., fluid outlet 114, outflow line 106, or another portion) and provide a flow signal indicative of the flow rate and/or pressure to control circuitry 120. In addition to or instead, sensor 121 may be configured to measure a position of impeller 118 (e.g., a position relative to pump housing 117) and/or an electrical characteristic of pump 104 (e.g., a current and/or voltage demand) and provide a flow signal indicative of the position and/or the electrical characteristic to control circuitry 120. In some examples, sensor 121 is configured to generate a flow signal representative of a mechanical wave generated by MCS device 102. The mechanical wave may, for example, include an acoustic wave, a mechanical vibration, an electrical field, or another parameter representative of a mechanical wave generated by an operation of MCS device 102. Control circuitry 120 may be configured to evaluate the flow signal to indicate MCS device 102 is producing a pulsating flow (e.g., based on the variance and/or oscillation of a wave form exhibited by the flow signal). In examples, control circuitry 120 may evaluate the pulsating flow generated by MCS device 102 by evaluating a plurality of frequency peaks, periods, or other characteristics of the wave form of the flow signal.

In the example shown in FIG. 1, the one or more components of system 100 are powered by a power source 122. Power source 122 may be, for example, one or more batteries, or another power source configured to deliver electrical power to control circuitry 120, pump 104, as well as other components of system 100. In examples, power source 122 is separately housed from control circuitry 120. Power source 122 may be electrically coupled to control circuitry 120 by a power cord 124. In the illustrated example, control circuitry 120 and power source 122 are removably attached to a carrier 126. Carrier 126 may be configured to be wearable by patient 103, such that patient 103 may remain ambulatory while using medical system 100.

In some examples, as shown in FIG. 1, medical system 100 includes a monitor 128, a computing device 130, and user interface 132. In examples, monitor 128 includes sensing circuitry configured to generate a signal indicative of a physiological parameter of a patient (e.g., a cardiac signal such as an ECG, ECM, an activity level of patient 103, a respiratory rate, a mechanical wave of heart 101, an electrical field of heart 101, or another physiological parameter). Monitor 128 is configured to communicate the signal indicative of the physiological parameter to another device, such as computing device 130 (e.g., via a wired or wireless communication link 134) and/or control circuitry 120. In examples, computing device 130 is configured to communicate with control circuitry 120 via, for example, a wired or wireless link 136. Control circuitry 120 may be configured to alter an operation of pump 104 based on the indicative signal from monitor 128 and/or a communication from computing device 130.

User interface 132 is communicatively coupled to computing device 130 via a wired or wireless link 138. User interface 132 may be configured to communicate information indicative of the operation of pump 104 or another component of medical system 100 to a user (e.g., patient 103, a caregiver, and/or a clinician) or another entity, such as a remote server system. In addition, user interface 132 can be configured to receive user input, such as user input requesting a change to an operation of pump 104.

In some examples, medical system 100 is configured to cause pump 104 to control or alter a pressure range exhibited by the pulsations of the pulsating flow based on a communication from monitor 128 and/or computing device 130. For example, medical system 100 may be configured to cause pump 104 to control or alter a wave period of the pulsating flow based on a communication from monitor 128 and/or computing device 130. In some examples, monitor 128 is configured to detect a pump thrombosis within pump 104 and communicate a thrombosis signal to computing device 130 and/or control circuitry 120. Control circuitry 120 may be configured to alter the operation of pump 104 based on the thrombosis signal to, for example, generate a mechanical vibration of impeller 118 to attempt to clear the pump thrombosis. Further, although shown in FIG. 1 as separate devices, in other examples, one or more of monitor 128, computing device 130, and/or user interface 132 may be included in the same device. Monitor 128, computing device 130, and/or user interface 132 may be portable, e.g., able to be carried on or implanted in patient 103 to, for example, enable patient 103 to remain ambulatory while using medical system 100.

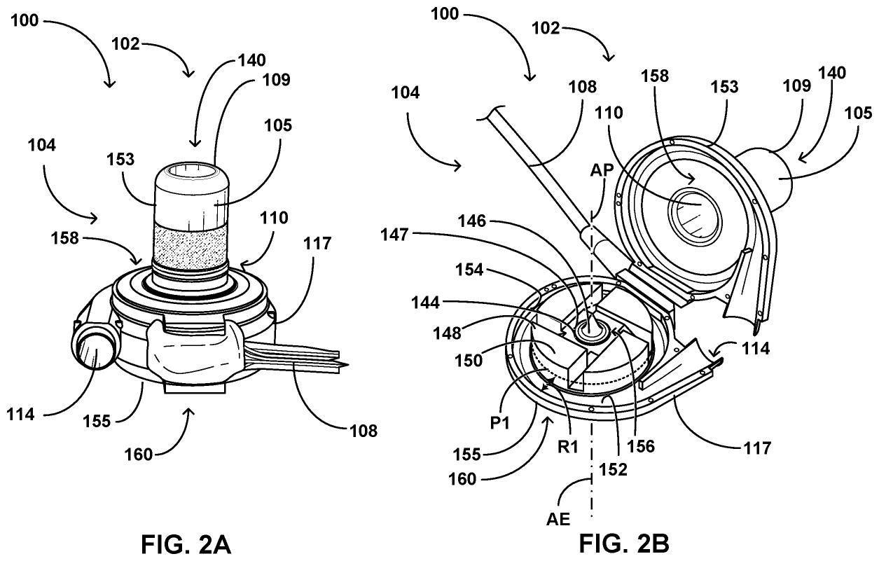

FIG. 2A schematically illustrates a perspective view of an example pump 104 with pump housing 117 in a first configuration. FIG. 2B illustrates the example pump 104 with pump housing 117 is a second configuration, in which pump housing 117 is open to illustrate impeller 118 positioned within pump housing 117. Pump housing 117 defines fluid inlet 110 and fluid outlet 114. Also shown in FIGS. 2A and 2B is inflow line 105, which defines a lumen 140 (“inflow line lumen 140”) and MCS inlet 109 opening to inflow line lumen 140. Fluid inlet 110 may be fluidically coupled to MCS inlet 109 through inflow line lumen 140. In examples, MCS device 102 is configured to receive a blood flow from heart 101 (FIG. 1) via inflow MCS inlet 109 and deliver the blood flow to pump 104 via inflow line lumen 140. Inflow line 105 can be integrally formed with pump housing 117 or can be physical separate from and mechanically connected to pump housing 117.

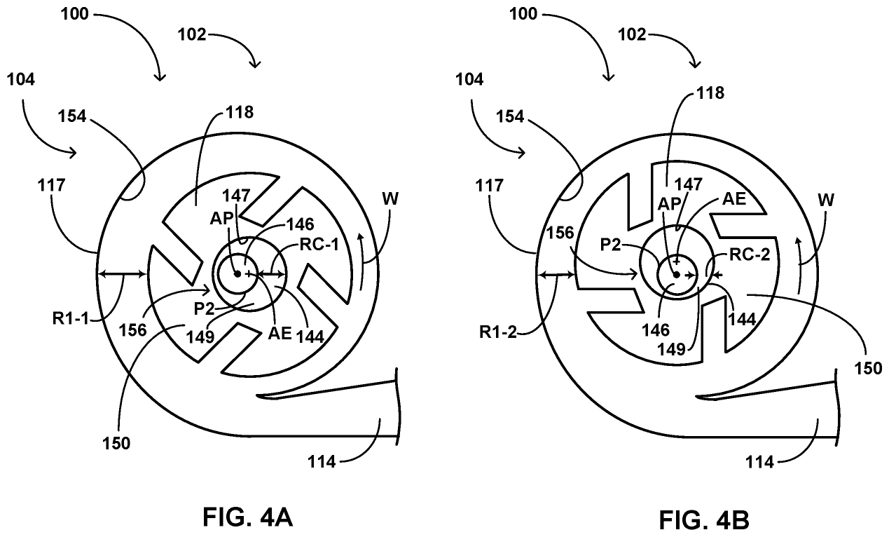

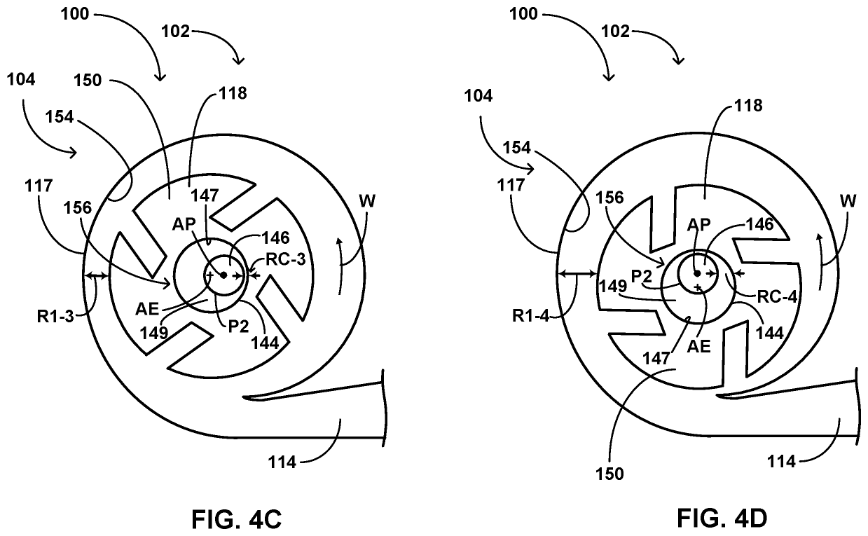

Pump 104 is configured to impart energy (e.g., a velocity) to a blood flow flowing from fluid inlet 110 to fluid outlet 114 when impeller 118 rotates within pump housing 117 (e.g., in the first configuration of FIG. 2A). Impeller 118 includes an impeller eye 144 configured to substantially surround (e.g., surround a majority of) a post 146 when impeller 118 rotates within pump housing 117. In examples, impeller 118 includes an inner surface 147 (“impeller inner surface 147”) forming the boundary of impeller eye 144. Impeller eye 144 and impeller inner surface 147 may define a volume 149 (“eye volume 149”) (FIGS. 4A-4D) configured to receive post 146 when impeller eye 144 substantially surrounds post 146. Post 146 is positioned within pump housing 117 and can be mechanically supported by pump housing 117. Post 146 can be integrally formed with pump housing 117 or physically separate from and mechanically connected to pump housing 117.

Pump 104 may be configured to impart energy to the blood flow by increasing a velocity of the blood flow, increasing a static pressure of the blood flow, and/or increasing a dynamic pressure of the blood flow. In examples, pump 104 (e.g., impeller 118) is configured to increase a velocity of the blood flow and then decrease the velocity of the blood flow (e.g., using pump housing 117) to cause an increase in the static pressure of the blood flow. Pump 104 may be configured to impart kinetic energy to the blood flow using impeller 118 and convert the kinetic energy to pressure energy using pump housing 117.

Impeller 118 may be configured to impart energy to the blood flow by accelerating the blood flow toward an impeller outer perimeter P1 defined by impeller 118 as impeller 118 rotates. Impeller outer perimeter P1 may be defined by, for example, one or more vane tips of impeller 118, such as vane tip 148 of impeller vane 150, and the path vane tip 148 takes as impeller 118 rotates within pump housing 117. Impeller 118 may be configured such that rotational motion of impeller 118 generates centrifugal forces in a direction from impeller inner surface 147 toward impeller outer perimeter P1. Impeller 118 may be configured to impart the centrifugal forces to a fluid flowing from impeller eye 144 toward impeller outer perimeter P1 (e.g., using one or vanes, such as vane 150), such that impeller 118 imparts energy to the fluid. Impeller 118 may comprise, for example, a radial impeller, a mixed flow impeller, an axial flow impeller, a peripheral impeller, a free flow impeller, or some other impeller configured to impart energy to a fluid flow.

Pump housing 117 is configured to fluidically couple fluid inlet 110 and fluid outlet 114 (e.g., when in the first configuration of FIG. 2A). Pump housing 117 may be configured to receive and direct the blood flow accelerated by impeller 118 toward fluid outlet 114. In examples, pump housing 117 is configured to increase a static pressure of the accelerated blood flow as the blood flow flows toward fluid outlet 114. In examples, pump housing 117 defines a volume 152 fluidically coupling the fluid inlet and the fluid outlet. Volume 152 may define, for example, a volute or a diffuser. In examples, pump housing 117 defines a boundary (e.g., a pressure boundary and/or fluid boundary) between volume 152 and an exterior of pump 104, such that MCS device 102 may more effectively generate a blood flow from MCS inlet 109 to MCS outlet 107. In examples, an inner surface 154 of pump housing 117 (“housing inner surface 154”) defines volume 152.

Pump housing 117 has any suitable configuration and is configured for implantation in a patient in some examples. In some examples, pump housing 117 includes an upper housing 153 configured to mechanically engage a lower housing 155 to define volume 152. Upper housing 153 and lower housing 155 may be configured to mechanically disengage to, for example, allow access to portions of volume 152 during assembly or for other reasons. In some examples, pump housing 117 is a unified body defining volume 152. Pump housing 117 may comprise, for example, titanium, a ceramic material, and/or another suitable biocompatible material. In examples, pump housing 117 comprises a non-magnetic material.

Pump 104 is configured to magnetically and/or hydrodynamically suspend impeller 118 within pump housing 117, such that impeller 118 maintains an outer radial clearance (e.g., the first outer radial clearance R1) between impeller 118 and housing inner surface 154 as impeller 118 imparts energy to the blood flow. Pump 104 may be configured to maintain an outer radial clearance between impeller 118 and housing inner surface 154 circumferentially around the impeller outer perimeter P1 as impeller 118 rotates.

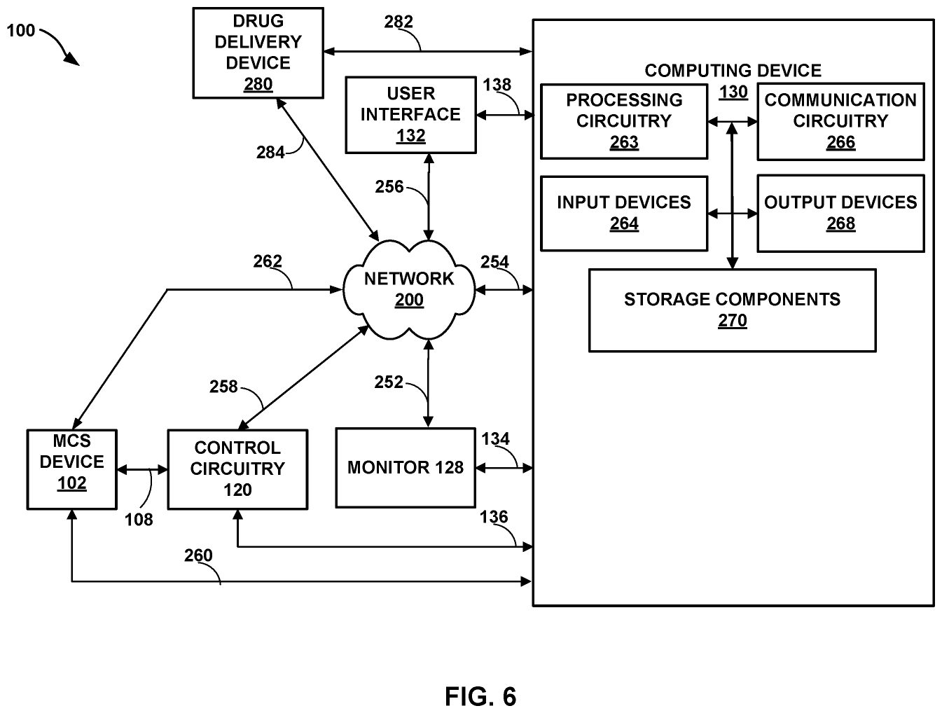

Pump 104 includes a magnetic bearing 156 configured to establish an inner radial clearance between impeller 118 and post 146 as impeller 118 rotates. In examples, magnetic bearing 156 includes an inner ring (not shown in FIGS. 2A and 2B) mechanically supported by post 146 and configured to magnetically interact with an outer ring (not shown in FIGS. 2A and 2B) mechanically supported by impeller 118 to maintain the inner radial clearance. Magnetic bearing 156 may be configured to cause magnetic forces between the inner ring and the outer ring which vary around an inner perimeter (e.g., inner perimeter P2 (FIGS. 4A-4D)) defined by post 146 and/or an inner ring of magnetic bearing 156 (e.g., inner ring 166 (FIG. 6)).

Pump 104 also includes a stator 158 configured to cause impeller 118 to rotate within pump housing 117. In examples, pump 104 defines a second stator 160. Stator 158 and/or second stator 160 may be mechanically supported by pump housing 117 and can be integrally formed with pump housing 117 or separate from and attached to pump housing 117. In examples, impeller 118 and stator 158 and/or second stator 160 are configured to magnetically interact to cause impeller 118 to rotate within pump housing 117. Impeller 118 and stator 158 and/or stator 160 may define a motor (e.g., a brushless DC motor) and a motor magnetic field configured to exert a torque on impeller 118 to cause impeller 118 to rotate within pump housing 117. Driveline 108 may be configured to provide electrical power to pump 104 to power magnetic bearing 156, stator 158, second stator 160, and/or other components of pump 104.

Pump 104 is configured to generate a pulsating blood flow (e.g., at fluid outlet 114) when impeller 118 rotates within pump housing 117 to impart energy to the blood flow at a given rate of rotation (e.g., pump speed). In examples, pump 104 is configured to generate the pulsating flow by at least causing a displacement defined by an outer radial clearance (e.g., defined by first outer radial clearance R1) to vary as impeller 118 rotates within pump housing 117. In some examples, pump 104 is configured to cause first outer radial clearance R1 to cyclically vary over a range between and including a minimum first radial clearance and a maximum first radial clearance as impeller 118 rotates within pump housing 117. For example, pump 104 may be configured such that the varying magnetic forces caused by the bearing magnetic field and/or fluid momentum forces imparted to impeller 118 cause an eye axis AE defined by impeller 118 to translate around (e.g., orbit) a post axis AP when impeller 118 rotates around eye axis AE. Post 146 may define post axis AP extending through post 146 and impeller eye 144 may define an eye axis AE extending through impeller eye 144. Eye axis AE may be substantially parallel (e.g., parallel or nearly parallel to the extent permitted by manufacturing tolerances) to post axis AP in some examples. Pump 104 may be configured to cause impeller 118 to rotate around eye axis AE (e.g., as an axis of rotation) when impeller 118 rotates within pump housing 117. Translation of the eye axis AE around post axis AP as impeller 118 rotates may cause first outer radial clearance R1 to cyclically vary, such that impeller 118 generates the pulsating flow.

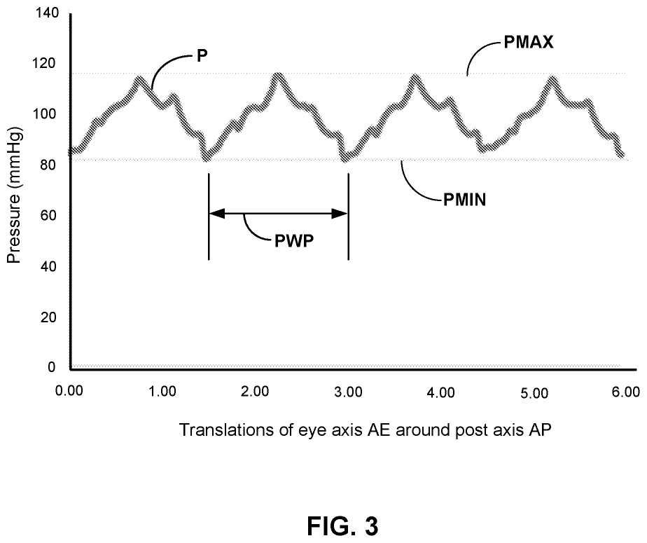

FIG. 3 illustrates an example of a pressure waveform P which may be generated when pump 104 provides the pulsating flow. FIG. 3 illustrates exemplary pressures of pressure waveform P, however pressure waveform P may exhibit any pressures sufficient for the operation of MCS device 102. Pump 104 may be configured to generate the pressure waveform P within, for example, pump housing 117, outflow line 106, and/or within some other portion of MCS device 102.

Pressure waveform P may substantially oscillate over a range defined between a maximum pressure PMAX and a minimum pressure PMIN. Pressure waveform P may define a pressure wave period PWP as pressure waveform P substantially oscillates over a range defined between a maximum pressure PMAX and a minimum pressure PMIN. In examples, pressure waveform P substantially oscillates over the range between maximum pressure PMAX and minimum pressure PMIN as the eye axis AE translates around (e.g., substantially orbits) the post axis AP. In some examples, pressure waveform P substantially oscillates over the range in a substantially synchronized manner with the translations of the eye axis AE around the post axis AP, such that a full translation of the eye axis AE around the post axis AP (labeled 1.00, 2.00, 3.00, etc. in FIG. 3) causes pressure waveform P to substantially oscillates over the range between maximum pressure PMAX and minimum pressure PMIN. In some examples, a rotation of impellor 118 around eye axis AE is synchronized with the translation of the eye axis AE around the post axis AP. For example, in some examples, for each full translation of eye axis AE around post axis AP, impeller 118 may complete a substantially full rotation around eye axis AE.

FIGS. 4A-4D schematically illustrate impeller 118 rotating around eye axis AE (e.g., rotating with eye axis AE as an axis of rotation) as eye axis AE translates around (e.g., orbits) post axis AP. In FIGS. 4A-4D, eye axis AE and post axis AP are depicted perpendicular to the page. FIGS. 4A-4D depict rotation of impeller 118 around eye axis AE as a sequence, such that FIG. 4B is temporally subsequent to the position depicted at FIG. 4A, FIG. 4C is temporally subsequent to the position depicted at FIG. 4B, and FIG. 4D is temporally subsequent to the position depicted at FIG. 4C. Magnetic bearing 156 generates magnetic forces which vary around inner perimeter P2 surrounding post 146. Impeller 118 rotates in a rotational direction W to impart energy to a blood flow by accelerating the blood flow toward housing inner surface 154 of pump housing 117 using, for example, impeller vane 150.

Pump 104 is configured such the varying magnetic forces of magnetic bearing 156 and/or fluid momentum forces on impeller 118 (e.g., as impeller 118 accelerates the blood flow toward housing inner surface 154) causes eye axis AE to translate around post axis AP when impeller 118 rotates around eye axis AE. For example, as impeller 118 from the impeller position depicted at FIG. 4A to the impeller position depicted at FIG. 4B, eye axis AE may translate around post axis AP from the position depicted at FIG. 4A to the position depicted at FIG. 4B. As impeller 118 rotates from the impeller position depicted at FIG. 4B to the impeller position depicted at FIG. 4C, eye axis AE may translate around post axis AP from the position depicted at FIG. 4B to the position depicted at FIG. 4C. As impeller 118 rotates from the impeller position depicted at FIG. 4C to the impeller position depicted at FIG. 4D, eye axis AE may translate around post axis AP from the position depicted at FIG. 4C to the position depicted at FIG. 4D. Impeller 118 may subsequently rotate from the impeller position depicted at FIG. 4D to substantially return to the impeller position depicted at FIG. 4A, such that eye axis AE translates around post axis AP from the position depicted at FIG. 4D to the position depicted at FIG. 4A. Hence, pump 104 may be configured to cause eye axis AE to translate around (e.g., orbit) post axis AP as impeller 118 rotates around eye axis AE to impart energy to a blood flow within pump housing 117.

A first outer radial clearance R1 (e.g., R1-1 (FIG. 4A), R1-2 (FIG. 4B), R1-3 (FIG. 4C), and/or R1-4 (FIG. 4D)) between impeller 118 and post 146 varies in magnitude as impeller 118 rotates. First outer radial clearance R1 is measured at one location of pump 104, such that it is measured at the same location in FIGS. 4A-4D. In some examples, pump 104 is configured such that the translation of eye axis AE around post axis AP causes the first outer radial clearance R1 to cyclically vary from a maximum first radial clearance (e.g., radial clearance R1-1 (FIG. 4A)) to a minimum first radial clearance (e.g., radial clearance R1-3 (FIG. 4C)) as eye axis AE translates around post axis AP. For example, pump 104 may be configured to define a second outer radial clearance (e.g., radial clearance R1-2 (FIG. 4B)) less than the maximum first radial clearance and greater than the minimum first radial clearance as impeller 118 rotates. Pump 104 may be configured to define the minimum first radial clearance (e.g., radial clearance R1-3 (FIG. 4C)) as impeller 118 rotates. Pump 104 may be configured to define a fourth outer radial clearance (e.g., radial clearance R1-4 (FIG. 4D)) greater than the minimum first radial clearance and less than the maximum first radial clearance as impeller 118 rotates. In examples, pump 104 is configured to cause impeller 118 and pump housing 117 (e.g., housing inner surface 154) to define the maximum first radial clearance, the second outer radial clearance, the minimum first radial clearance, and the fourth outer radial clearance substantially cyclically as impeller 118 rotates around eye axis AE. That is, as eye axis AE translates around post axis AP, impeller 118 moves such that it defines, relative to post 146, The maximum first radial clearance (e.g., R1-1), the second outer radial clearance (e.g.) R1-2, the minimum first outer radial clearance (e.g., R1-3), and the fourth outer radial clearance (e.g., R1-4).

The variation of the first outer radial clearance R1 over the range between and including the maximum first radial clearance and the minimum first radial clearance may cause impeller 118 to generate a pulsating flow as impeller 118 imparts energy to the blood flow within pump housing 117.

The translation of eye axis AE around post axis AP as impeller 118 rotates may cause an inner radial clearance RC between post 146 and impeller inner surface 147 (defining eye 144) to cyclically vary from a maximum inner radial clearance (e.g., radial clearance RC-1 (FIG. 4A) to a minimum inner radial clearance (e.g., RC-3 (FIG. 4C)). For example, pump 104 may be configured to define a second inner radial clearance (e.g., radial clearance RC-2 (FIG. 4B)) less than the maximum inner radial clearance and greater than the minimum inner radial clearance as impeller 118 rotates. Pump 104 may be configured to define the minimum inner radial clearance (e.g., radial clearance RC-3 (FIG. 4C)) as impeller 118 rotates in the rotational direction W. Pump 104 may be configured to define a fourth inner radial clearance (e.g., radial clearance RC-4 (FIG. 4D)) greater than the minimum inner radial clearance and less than the maximum inner radial clearance as impeller 118 rotates. In examples, pump 104 is configured to cause impeller 118 and post 146 to define the maximum inner radial clearance, the second inner radial clearance, the third inner radial clearance, and the minimum inner radial clearance substantially cyclically as impeller 118 rotates around eye axis AE. That is, as eye axis AE translates around post axis AP, impeller 118 moves such that it defines, relative to post 146, the maximum inner radial clearance (e.g., RC-1), the second inner radial clearance (e.g., RC-2), the minimum first inner radial clearance (e.g., RC-3), and the fourth inner radial clearance (e.g., RC-4). Inner radial clearance RC (e.g., RC-1, RC-2, RC-3, and RC-4) may be measured at one location of pump 104, such that it is measured at the same location in FIGS. 4A-4D.

In examples, pump 104 is configured such that the inner radial clearance RC between impeller 118 and post 146 varies proportionally to the first radial clearance R1 between the impeller and the housing. In examples, pump 104 defines the inner radial clearance RC in a direction substantially parallel to the direction of first outer radial clearance R1. In some examples, the first outer radial clearance R1 defines a first displacement vector in a first direction, and the inner radial clearance RC defines a second displacement vector in the first direction.

In examples, the varying inner radial clearance RC causes eye volume 149 to describe a substantially non-uniform shape with respect to post axis P as impeller 118 rotates around eye axis AE and eye axis AE translates around post axis P. For example, eye volume 149 may be defined by a plurality of outer radii passing defined from the post axis AP to impeller inner surface 147 and passing through inner perimeter P2 of post 146. The plurality of outer radii may define a varying radial displacement between post axis AP and impeller inner surface 146 around the inner perimeter 146, such that eye volume 149 describes a substantially non-uniform shape with respect to post axis AP. Magnetic bearing 156 may be configured to cause eye volume 149 to describe the substantially non-uniform shape as impeller 118 rotates around eye axis AE to impart energy to a fluid.

As used herein, when impeller 118 rotates around eye axis AE, this means that impeller 118 rotates as a fixed body around an axis of rotation. The axis of rotation may be substantially defined by and/or coincident with eye axis AE. When eye axis AE translates around post axis AP, this may mean that some portion of eye axis AE defines a closed perimeter around post axis AP as eye axis AE translates around post axis AP. In some examples, eye axis AE is substantially parallel with post axis AP when eye axis AE translates around post axis AP, although this is not required. The magnitudes of the maximum first radial clearance and the minimum first radial clearance may vary as impeller 118 proceeds from one rotation around eye axis AE to a subsequent rotation around eye axis AE. Hence, impeller 118 and pump housing 117 are configured to define an initial maximum first radial clearance and an initial minimum first radial clearance during an initial revolution of impeller 118 around eye axis AE, and define a subsequent maximum first radial clearance and a subsequent minimum first radial clearance during a subsequent revolution of impeller 118 around eye axis AE. The initial maximum first radial clearance may define a displacement (e.g., a length) different from the subsequent maximum first radial clearance and/or the initial minimum first radial clearance may define a displacement (e.g., a length) different from the subsequent minimum first radial clearance.

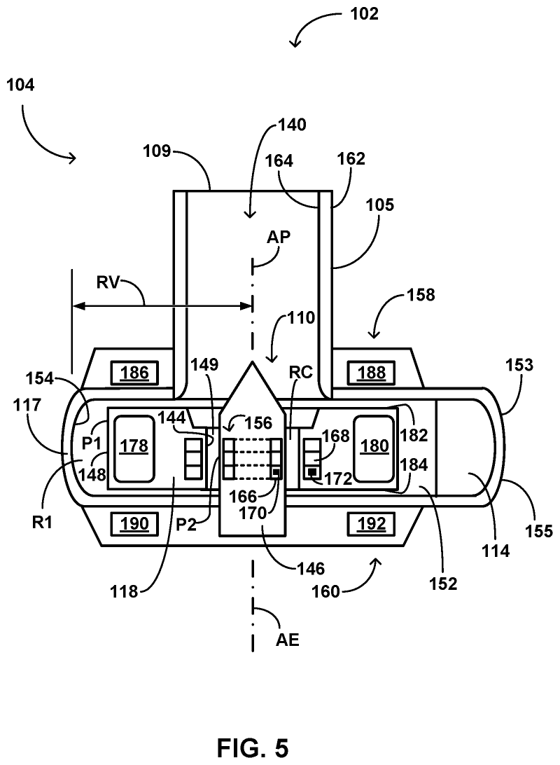

FIG. 5 illustrates a schematic cross-sectional view of example blood pump 104 configured to produce a pulsating flow as a blood flow flows from fluid inlet 110 to fluid outlet 114. The cross-section is taken through a center of pump housing 117, e.g., along post axis AP. Pump housing 117 is configured to house the components of pump 104. In examples, pump housing 117 includes upper housing 153 and lower housing 155 configured to mechanically engage to define volume 152. Post 146 is mechanically supported by pump housing 117 and defines post axis AP. Volume 152 may defines a radius RV between post axis AP and housing inner surface 154 that, in which example shown in FIG. 5, increases progressively (e.g., in a clockwise of counter-clockwise direction) around post axis AP to fluid outlet 114. Upper housing 153 and lower housing 155 may be configured to mechanically engage to define fluid outlet 114.

Inflow line 105 is configured to receive a blood flow via MCS inlet 109 and provide the provide the blood flow to impeller 118 via inflow line lumen 140 and fluid inlet 110. In examples, pump housing 117 mechanically supports inflow line 105. Pump housing 117 may mechanically support inflow line 105 such that post axis AP extends at least partially through fluid inlet 110 and/or inflow line lumen 140. In examples, pump housing 117 (e.g., upper housing 153 and lower housing 155) and inflow fine 105 may be fixedly connected to define a continuous, enclosed flow path from MCS inlet 109 to fluid outlet 114. In examples, inflow line 105 includes an outer surface 162 (“inflow outer surface 162”) and an inner surface 164 (“inflow inner surface 164”) opposite inflow outer surface 162. Inflow inner surface 164 may define inflow line lumen 140.

Impeller 118 is fully or partially enclosed by pump housing 117. Impeller 118 is configured to receive a blood flow via fluid inlet 110 and impart energy to the blood flow as the blood flow flows from fluid inlet 110 to fluid outlet 114. Impeller 118 is configured to rotate around eye axis AE defined by impeller eye 144 when impeller 118 is positioned within pump housing 117. Eye axis AE extends through eye volume 149 defined by impeller eye 144. In examples, impeller 118 is configured such that eye volume 149 receives post 146 when impeller 118 is positioned within pump housing 117, such that impeller eye 144 substantially surrounds post 146. In examples, impeller 118 is configured to receive a blood flow (e.g., from fluid inlet 110) into eye volume 149 and rotate around eye axis AE to impart energy by accelerating the blood flow housing inner surface 154. Impeller 118 is configured to rotate to impart centrifugal forces to the blood flow flowing from impeller eye 144 toward housing inner surface 154.

Pump 104 includes a magnetic bearing 156 configured to establish and/or maintain the inner radial clearance RC between impeller 118 (e.g., impeller inner surface 147) and post 146 (e.g., inner perimeter P2). Magnetic bearing 156 may be configured to generate a bearing magnetic field which generate magnetic forces between impeller 118 and post 146. Magnetic bearing 156 may be configured such that the magnetic forces (e.g., attractive or repulsive) maintain the inner radial clearance RC. In examples, magnetic bearing 156 is configured to cause the magnetic forces to act between impeller 118 and post 146 around some portion of or substantially all of a perimeter defined around post axis AP and/or eye axis AE, such that a first force vector acting in a first direction (e.g., on one of impeller 118 or post 146) substantially opposes a second force vector acting in a second direction (e.g., on the one of impeller 118 or post 146) opposite the first direction. For example, the first force vector and the second force vector may act through a closed perimeter in a plane substantially perpendicular to post axis AP. The opposition of the first force vector and the second force vector may act to establish and/or maintain the inner radial clearance RC between impeller 118 (e.g., impeller inner surface 147) and post 146.

In the example shown in FIG. 5, magnetic bearing 156 includes an inner ring 166 and an outer ring 168 configured to establish and/or maintain the inner radial clearance RC. The bearing magnetic field generated by magnetic bearing 156 causes inner ring 166 to magnetically interact with outer ring 168. Magnetic bearing 156 may be configured such that the magnetic interaction between inner ring 166 and outer ring 168 causes the magnetic force between impeller 118 and post 146. In some examples, magnetic bearing 156 is configured to cause inner ring 166 to generate the magnetic field causing inner ring 166 to magnetically interact with outer ring 168. In other examples, magnetic bearing 156 is configured to cause outer ring 168 to generate the magnetic field causing inner ring 166 to magnetically interact with outer ring 168. Inner ring 166 may be configured to transmit some portion of the magnetic force to post 146. Outer ring 168 may be configured to transmit some portion of the magnetic force to impeller 118. In examples, inner ring 166 is mechanically supported by and/or integral with post 146. Outer ring 168 may be mechanically supported by and/or integral with impeller 118.

Magnetic bearing 156 is configured to cause the magnetic forces to vary around the perimeter defined around post axis AP and/or eye axis AE. In examples, magnetic bearing 156 may be configured to cause inner radial clearance RC to vary around inner perimeter P2. Magnetic bearing 156 may be configured such that the magnetic forces cause inner radial clearance RC to vary such that eye axis AE is substantially offset from post axis AP when the magnetic forces establish and/or maintain the inner radial clearance RC.

In some examples, magnetic bearing 156 is configured as a passive magnetic bearing including one or more permanent magnets. For example, inner ring 166 and/or the outer ring 168 may include a permanent magnet configured to cause magnetic forces between inner ring 166 and outer ring 168. In some examples, one of inner ring 166 or outer ring 168 includes a permanent magnet and the other of inner ring 166 or outer ring 168 includes a ferromagnetic material configured to interact with a magnetic field generated by the permanent magnet. The permanent magnet may comprise, for example, iron, a steel, nickel, platinum, cobalt, samarium, boron, and/or other materials suitable for permanent magnetization. The ferromagnetic material may comprise, for example, iron, nickel, cobalt, a steel, a “soft iron” having a relatively low coercivity, or another suitable material. The permanent magnet may be configured to cause the magnetic forces between post 146 and impeller 118 to vary around the perimeter defined around post axis AP and/or eye axis AE. For example, the permanent magnet may be configured to generate a magnetic field exhibiting a varying magnetic flux density around the perimeter defined around post axis AP and/or eye axis AE. The varying magnetic flux density may cause the magnetic forces between post 146 and impeller 118 to vary around the perimeter defined around post axis AP and/or eye axis AE. In some examples, the permanent magnet defines a structural feature (e.g., a notch and/or face in the permanent magnet) configured to cause the magnetic flux density and/or the magnetic forces to vary.

In other examples, magnetic bearing 156 is configured as an active magnetic bearing including one or more electromagnets. For example, inner ring 166 and/or the outer ring 168 may include an electromagnet configured to cause magnetic forces (attractive or repulsive) between inner ring 166 and outer ring 168. In examples, one of inner ring 166 or outer ring 168 includes an electromagnet and the other of inner ring 166 or outer ring 168 includes a ferromagnetic material configured to interact with a magnetic field generated by the electromagnet. The electromagnet may be configured to cause the magnetic forces between post 146 and impeller 118 to vary around the perimeter defined around post axis AP and/or eye axis AE. In examples, the electromagnet is configured to generate a magnetic field exhibiting a varying magnetic flux density around the perimeter defined around post axis AP and/or eye axis AE. The varying magnetic flux density may cause the magnetic forces between post 146 and impeller 118 to vary around the perimeter defined around post axis AP and/or eye axis AE.

In some examples, magnetic bearing 156 includes a winding configured to generate the magnetic field of the electromagnet. For example, inner ring 166 may include a winding 170 configured to cause inner ring 166 to generate a magnetic field. Outer ring 168 may include a winding 172 configured to cause outer ring 168 to generate a magnetic field. In examples, winding 170, 172 is configured to generate a magnetic field causing the magnetic forces between post 146 and impeller 118 to vary around the perimeter defined around post axis AP and/or eye axis AE.

As discussed, pump 104 is configured such that eye axis AE translates around (e.g., orbits) post axis AP when impeller 118 rotates around eye axis AE to impart energy to a blood flow within pump housing 117. The translation of eye axis AE around post axis AP may cause the inner radial clearance RC to cyclically vary from a maximum inner radial clearance (e.g., radial clearance RC-1 (FIG. 4A) to a minimum inner radial clearance (e.g., RC-3 (FIG. 4C)). Pump 104 may be configured such that a magnetic field (e.g., a magnetic field strength) generated by winding 170, 172 substantially controls the maximum inner radial clearance and/or the minimum inner radial clearance as eye axis AE translates around post axis AP. Pump 104 may be configured such that a pressure range exhibited by the pulsating flow (e.g., at fluid outlet 114) is controllable based on the difference between the minimum inner radial clearance and the maximum inner radial clearance. Hence, in examples, control circuitry 120 is configured to control a pressure range exhibited by the pulsating flow by at least controlling the maximum inner radial clearance and/or the minimum inner radial clearance caused by the magnetic field generated by winding 170, 172. Control circuitry 120 may be configured to alter the pressure range exhibited by the pulsating flow by at least altering the magnetic field through, for example, altering electric power supplied to winding 170, 172.

In similar manner, the translation of eye axis AE around post axis AP may cause the first outer radial clearance R1 to cyclically vary from a maximum first radial clearance (e.g., radial clearance R1-1 (FIG. 4A)) to a minimum first radial clearance (e.g., radial clearance R1-3 (FIG. 4C)). In some examples, pump 104 is configured such that the magnetic field (e.g., the magnetic field strength) generated by winding 170, 172 substantially controls the maximum first radial clearance and/or the minimum first radial clearance. Pump 104 may be configured such that the pressure range exhibited by the pulsating flow is controllable based on the difference between the minimum first radial clearance and the maximum first radial clearance, such that control circuitry 120 may control the pressure range of the pulsating flow by at least controlling the maximum first radial clearance and/or the minimum first radial clearance caused by the magnetic field generated by winding 170, 172.

In examples, control circuitry 120 is configured to control the magnetic field generated by winding 170, 172. Control circuitry 120 may be configured to control the electric power supplied (e.g., via driveline 108 (FIG. 1)) to winding 170, 172 to control the magnetic field. For example, control circuitry 120 may be configured to control the magnetic field generated by winding 170, 172 to control and/or alter the maximum inner radial clearance, minimum inner radial clearance, minimum first radial clearance, and/or the maximum first radial clearance based on a signal (e.g., from monitor 128 (FIG. 1)) representative of a physiological parameter of heart 101. Hence, control circuitry 120 may be configured to alter the characteristics of the pulsating flow depending on the needs of the patient.

In some examples, magnetic bearing 156 may be configured as a hybrid magnetic bearing including both one or more electromagnets and one or more permanent magnets. For example, inner ring 166 may include a permanent magnet, an electromagnet, or a permanent magnet and an electromagnet. Outer ring 168 may include a permanent magnet, an electromagnet, or a permanent magnet and an electromagnet. Control circuitry 120 may be configured to control the power supply to winding 170 to control the magnetic field generated by inner ring 166, control the power supply to winding 172 to control the magnetic field generated by outer ring 168, or control both the first power supply to winding 170 and the second power supply to winding 172.

Pump 104 may include stator 158 and/or second stator 160 configured to cause impeller 118 to rotate around eye axis AE to impart energy to a blood flow within pump housing 117. Stators 158, 160 may mechanically supported by pump housing 117. Stators 158, 160 may be configured to generate a motor magnetic field to cause the rotation of impeller 118. In examples, the motor magnetic field exerts a torque on impeller 118 causing the rotation of impeller 118. In examples, stators 158, 160 are configured to generate a motor magnetic field rotating substantially around post axis PA and/or eye axis AE to cause impeller 118 to rotate. Pump 104 may be configured such that a rotational speed of the motor magnetic field controls the rotational speed of impeller 118 around eye axis AE. In examples, stator 158 includes one or more field elements such as field element 186 and field element 188. Second stator 160 may include one or more field elements such as field element 190 and field element 192. Field element 186, 188, 190, 192 may include one or more windings configured to generate an electromagnetic field. The one or more windings may be configured to generate the motor magnetic field using AC electric power or DC electric power (e.g., supplied via driveline 108 (FIG. 1)). In some examples, field element 186, 188, 190, 192 includes one or more permanent magnets configured to generate a magnetic field.

Control circuitry 120 (FIG. 1) may be configured to control a rotational speed and/or magnetic field strength of the motor magnetic field to, for example, control a speed and/or generated torque of pump 104. For example, control circuitry 120 may control the electric power supplied to stator 158, 160 (e.g., via driveline 108) to control the rotational speed and/or magnetic field strength of the motor magnetic field. Control circuitry 120 may be configured to alter the characteristics of the motor magnetic field to alter the pulsating flow produced by pump 104. For example, pump 104 may be configured such that a pressure range and/or pressure wave period exhibited by the pulsating flow of pump 104 is controllable based on the speed of pump 104. Control circuitry 120 may be configured to control the pressure range and/or pressure wave period by at least controlling the speed of pump 104. In examples, control circuitry 120 is configured to control the motor magnetic field to alter a speed a pump 104 based on a signal (e.g., from monitor 128 (FIG. 1)) representative of a physiological parameter of patient 103. In some examples, control circuitry 120 is configured to alter a generated torque of pump 104 to, for example, cause a mechanical vibration to impeller 118 to clear a thrombosis within pump 104, or for other reasons. Hence, control circuitry 120 may be configured to alter the characteristics of the pulsating flow depending on the needs of the patient.

Pump 104 may be configured such that the motor magnetic field magnetically interacts with impeller 118 to cause impeller 118 to rotate around the eye axis. For example, impeller 118 may include one or more impeller pole pieces such as pole piece 178 and pole piece 180 configured to magnetically interact with the motor magnetic field. Pole piece 178, 180 may comprise, for example, a ferrous and/or ferromagnetic material. Pump 104 may be configured such that the magnetic interaction of impeller 118 with the motor magnetic field exerts a torque on impeller 118 causing impeller 118 to rotate around eye axis AE. In some examples, pole piece 178, 180 includes a permanent magnet configured to magnetically interact with the motor magnetic field. Impeller 118 may mechanically support pole pieces such that when the motor magnetic field causes a torque on pole piece 178, 180 around eye axis AE, pole piece 178, 180 transmits at least some portion of the torque to impeller 118 to cause the rotation of impeller 118 around eye axis AE. In some examples, stator 158, 160 and pole piece 178, 180 operate substantially as a brushless DC motor (BLDM) to cause the rotation of impeller 118 around eye axis AE.

In examples, pump 104 is configured such that impeller 118 is suspended within pump housing 117 when impeller 118 rotates to impart energy to a blood flow within pump housing 117. Pump 104 may be configured to maintain an axial clearance between impeller 118 and pump housing 117 (e.g., a clearance in a direction substantially parallel to post axis AP and/or eye axis AE) when impeller 118 impart the energy to the blood flow. Pump 104 may be configured such that the motor magnetic field and/or hydrodynamic forces from the blood flow cause the axial displacement. In examples, rotation of impeller 118 causes some portion of the blood flow entering pump housing 117 via fluid inlet 110 to define one or more hydrodynamic bearings with impeller 118 to causes impeller 118 to axially suspend. Pump 104 may be configured such that the hydrodynamic bearing acts substantially as a thrust bearing for impeller 118 to, for example, assist impeller 118 in its rotation around eye axis AE.

For example, impeller 118 may define a first bearing surface 182 configured to allow a portion of the blood flow entering via fluid inlet 110 to flow between first bearing surface 182 and pump housing 117 when impeller 118 rotates around eye axis AE. Impeller 118 may define a second bearing surface 184 configured to allow a portion of the blood flow flowing into eye volume 149 to flow between second bearing surface 184 and pump housing 117 when impeller 118 rotates around eye axis AE. The blood flow flowing between pump housing 117 and first bearing surface 182 and/or second bearing surface 184 may support impeller 118 during rotation and help keep impeller 118 out of contact with pump housing 117 (e.g., upper housing 153 and/or lower housing 155). In examples, the blood flow flowing between pump housing 117 and first bearing surface 182 and/or second bearing surface 184 may substantially act as a thrust bearing for impeller 118 to resist axial loads on impeller 118 arising from magnetic forces between stator 158, 160 and pole piece 178, 180.

FIG. 6 is a functional block diagram illustrating an example medical system 100 that includes MCS device 102. As shown in FIG. 6, monitor 128, computing device 130, user interface 132, and control circuitry 120 are optionally communicatively coupled to a network 200. In some examples, fewer components (e.g., only computing device 130) may be coupled to network 200. Network 200 represents any public or private communication network, for instance, based on Bluetooth, WiFi®, a proprietary protocol for communicating with IMDs, or other types of networks for transmitting data between computing systems, servers, and computing devices, both implanted within and external to a patient. Monitor 128, computing device 130, user interface 132, control circuitry 120, and MCS device 102 may each be operatively coupled to network 200 using respective network links 252, 254, 256, 258, and 262. Network links 252, 254, 256, 258, 262 may be any type of network connections, such as wired or wireless connections as discussed above.

Network 200 may provide selected devices, such as monitor 128, computing device 130, user interface 132, control circuitry 120, and MCS device 102 with access to the Internet, and may enable monitor 128, computing device 130, user interface 132, control circuitry 120, and MCS device 102 to communicate with each other. For example, rather than communicating via link 134, monitor 128 and computing device 130 may communicate via network links 252 and 254. Rather than communicating via link 136, computing device 130 and control circuitry 120 may communicate via network links 254 and 258. In some examples, computing device 130 is configured to communicate with MCS device 102 via link 260, which may be configured similarly to links 252, 254, 256, 258, and 262 described above. In some examples, rather than communicating via link 260, MCS device 102 and computing device 130 may communicate via network links 262 and 254.

In some examples, computing device 130 is configured to send data to MCS device 102 and/or control circuitry 120, receive data from MCS device 102 and/or control circuitry 120, or both via network 200. For example, computing device 130 may send at least one of one or more signals (e.g., conditioned or unconditioned signals from monitor 128), MCS data, auxiliary cardiovascular data, and user data to MCS device 102 and/or control circuitry 120. Computing device 130 also may receive data from MCS device 102 and/or control circuitry 120 including, for example, stored signals, stored indications (e.g., from sensor 121), stored user data, notification data (e.g., regarding indications of, for example, an operation and/or condition of MCS device 102), algorithm data (e.g., to update or modify algorithms used by computing device 130 or control circuitry 120 to, for example, control MCS device 102), and the like. Computing device 130 may collect and analyze one or more signals, indications of MCS device 102 operation, MCS data, auxiliary cardiovascular data, and user data from at least one of computing device 130 and/or control circuitry 120 to notify patient 103 of another user (e.g., via user interface 132) of an operation of and/or indication from MCS device 102, such as a pump model, a pump of an identified age range, a pump with an identified operational history, a pump used in a patient with an identified medical history or treatment history, or the like.

In examples, computing device 130 includes storage components 270 to store data provided by monitor 128, control circuitry 120, sensor 121, and/or other components of medical system 100, or any combination thereof. Control circuitry 120 is configured to alter the operation of pump 104 based on the data provided by monitor 128 and/or control circuitry 120, track the data provided by monitor 128 and/or control circuitry 120 over time, or both, e.g., directly or under the control of computing device 130. In examples, computing device 130 may receive (e.g., from control circuitry 120) data associated with pump 104, such as but is not limited to, the age and model type of one or more components of pump 104, the age and usage of power source 122, the power consumption of pump 104, flow data associated with blood flow through pump 104, a temperature of pump 104, revolutions per minute of pump 104, user input data from user interface 132, or other data.

In some examples, monitor 128 is within or coupled to an implantable medical device (IMD). The IMD may include, but is not limited to, at least one of MCS device 102 or an insertable cardiac monitor. In some examples, monitor 128 may be within or coupled to a wearable device (e.g., an externally-wearable device) or other portable device, such as, for example, a mobile phone, patch, chest strap, or a Holter monitor. In examples, monitor 128 is configured to generate a signal (e.g., output) representative of a physiological parameter of patient 103 and provides the signal to computing device 130, control circuitry 120, user interface 132, and/or a remote server. In some examples, monitor 128 may be configured to condition the signal prior to providing the signal to computing device 130, control circuitry 120, user interface 132, and/or the remote server. Conditioning may include, but is not limited to, amplification, filtering, attenuation, isolation, and/or transformation such as Fast Fourier Transformation. In some examples, monitor 128 may provide an unconditioned signal to computing device 130, control circuitry 120, user interface 132, and/or the remote server. Computing device 130, control circuitry 120, user interface 132, and/or the remote server may condition the signal in some examples.

User interface 132 is configured to receive input from a user and/or communicate information to a user (e.g., patient 103, a caregiver, and/or a clinician) or another entity, such as a remote server system. User interface system may include any suitable user input devices, such as, but not limited to, a display, a keyboard, buttons, a touchscreen, a speaker, a microphone, a gyroscope, an accelerometer, a vibration motor, or the like. In examples, user interface 132 is configured to generate an alert based on a communication received from computing device 130, monitor 128, and/or control circuitry 120. In some examples, a display of user interface 132 may include a mobile device of the user. Similarly, computing device 130 includes one or more input components that receive tactile input, kinetic input, audio input, optical input, or the like from a user or another entity via user interface 132. In this way, user interface 132 may receive user input from a user and send user input to computing device 130 or control circuitry 120. For example, a user may provide user input to user interface 132, which communicates the user input to computing device 130 or control circuitry 120 to control an operation of MCS device 102.

In some, but not all, examples, medical system 100 includes drug delivery device 280. Drug delivery device 280 is configured to deliver any suitable drug therapy to patient 103. Drug delivery device may be communicatively coupled to computing device 130 via link 282. Additionally or alternatively, drug delivery device 280 may be operatively coupled to network 200 via network link 284. Link 282, 284 may be the same as or substantially similar to links 252, 254, 256, 258, 260, and 262, as discussed above, as discussed above. Computing device 130 may control drug delivery device 280 to deliver an intervention to a patient. For example, computing device 130 may control drug delivery device 280 to deliver or increase a rate of delivery of a thrombolytic agent to MCS device 102, such as into a blood stream of a patient or directly to a component of MCS device 102. The thrombolytic agent may be configured to breakup (e.g., at least partially dissolve) or dislodge a thrombus from MCS device 102 (e.g., medical pump 104). Medical system 100 may be communicatively coupled (e.g., connected) to one or more additional cardiovascular system monitoring devices. Cardiovascular system monitoring devices include, but are not limited to, pulse monitoring devices, blood oxygenation monitoring devices, blood pressure monitoring devices, prothrombin time monitoring devices, and additional user input devices.

Although computing device 130 of FIG. 6 is shown separate from monitor 128, user interface 132, and control circuitry 120, in some examples, computing device 130 may include one or more of monitor 128, user interface 132, and control circuitry 120. For example, rather than being coupled by links, computing device 130 and user interface 132 may form an integrated device, such as a mobile phone or a wearable medical device monitor. In one example approach, computing device 130 includes processing circuitry 263, one or more one or more input devices 264, communications circuitry 266, one or more output devices 268, and one or more one or more storage components 270. In some examples, computing device 130 may include additional components or fewer components than those illustrated in FIG. 6.

Control circuitry 120 and processing circuitry 263 may include various type of hardware, including, but not limited to, microprocessors, controllers, digital signal processors (DSPs), application specific integrated circuits (ASICs), field-programmable gate arrays (FPGAs), or equivalent discrete or integrated logic circuitry, as well as combinations of such components. The term “processing circuitry” and “control circuitry” may generally refer to any of the foregoing logic circuitry, alone or in combination with other logic circuitry, or any other equivalent circuitry. Control circuitry 120 and processing circuitry 263 may represent hardware that can be configured to implement firmware and/or software that sets forth one or more algorithms described herein. For example, processing circuitry 263 may be configured to implement functionality, process instructions, or both for execution within computing device 130 of processing instructions stored within storage components 270. In some examples, processing circuitry 263 includes processing circuitry of an IMD and/or other devices of medical system 100.

Input devices 264, in some examples, are configured to receive input from a user through tactile, audio, or video sources. Examples of input devices 264 include user interface 132, a mouse, a button, a keyboard, a voice responsive system, video camera, microphone, touchscreen, or any other type of device for detecting a command from a user. In some example approaches, user interface 132 includes all input devices 264 employed by computing device 130.

Computing device 130 may utilize communications circuitry 266 to communicate with external devices (e.g., monitor 128, control circuitry 120, user interface 132, and/or MCS device 102) via one or more networks, such as one or more wired or wireless networks. Communications circuitry 266 may include a communications interface, such as an Ethernet card, a radio frequency transceiver, cellular transceiver, a Bluetooth® interface card, USB interface, or any other type of device that can send and receive information. In some examples, computing device 130 utilizes communications circuitry 266 to wirelessly communicate with an external device such as a remote server system.

Computing device 130 may further include one or more output devices 268. Output devices 246, in some examples, are configured to provide output to a user using, for example, audio, video or tactile media. For example, output devices 246 may include user interface 132, a sound card, a video graphics adapter card, or any other type of device for converting a signal into an appropriate form understandable to humans or machines. In some example approaches, user interface 132 includes all output devices 246 employed by computing device 130.

One or more storage components 270 may be configured to store information within computing device 130 during operation. One or more storage components 270, in some examples, include a computer-readable storage medium or computer-readable storage device. In some examples, one or more storage components 270 include a temporary memory, meaning that a primary purpose of one or more storage components 270 is not long-term storage. One or more storage components 270, in some examples, include a volatile memory, meaning that one or more storage components 270 does not maintain stored contents when power is not provided to one or more storage components 270. Examples of volatile memories include random access memories (RAM), dynamic random-access memories (DRAM), static random-access memories (SRAM), and other forms of volatile memories known in the art. In some examples, one or more storage components 270 are used to store program instructions for execution by processing circuitry 263. One or more storage components 270, in some examples, are used by software or applications running on computing device 130 to temporarily store information during program execution. In some examples, one or more storage components 270 may further include one or more storage components 270 configured for longer-term storage of information. In some examples, one or more storage components 270 include non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

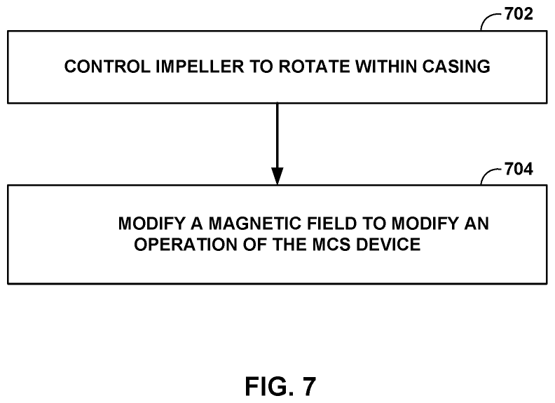

An example technique for connecting a generating a pulsating flow using a medical system is illustrated in FIG. 7. Although the technique is described mainly with reference to medical system 100 of FIGS. 1-5, and as being performed by control circuitry 120, the technique may be performed by another system and/or other control circuitry of system 100 alone or in combination with control circuitry 120.

The technique includes using controlling, by control circuitry 120, impeller 118 to rotate within pump housing 117 of pump 104 to impart energy to a fluid (702). Impeller 118 rotates around an eye axis AE extending through an impeller eye 144 defined by impeller 118 to impart the energy to the fluid. In examples, a magnetic bearing 156 establishes an inner radial clearance RC between impeller 118 and a post 146 mechanically supported by pump housing 117 as impeller 118 rotates to impart the energy to the fluid.

Magnetic bearing 156 may include an inner ring 166 mechanically supported by post 146 and an outer ring 168 mechanically supported by impeller 118. As discussed above, inner ring 166 and outer ring 168 may magnetically interact to establish the inner radial clearance RC. In examples, magnetic bearing 156 causes a first magnetic force between inner ring 166 and outer ring 168 in a first radial direction and a second magnetic force between inner ring 166 and outer ring 168 in a second direction substantially opposite the first direction, with the second magnetic force less than the first magnetic force. The differing magnetic forces caused by magnetic bearing 156 may cause the eye axis to translate around post axis when impeller 118 rotates to impart energy to the fluid, such that impeller 118 generates the pulsating flow. The magnetic interaction between inner ring 166 and outer ring 168 may cause the eye axis AE to translate around a post axis AP defined by post 146 when impeller 118 rotates around eye axis AE to impart the energy to the fluid. In examples, the magnetic interaction may cause the inner radial clearance RC to vary as impeller 118 imparts the energy to the fluid. In examples, the magnetic interaction may cause a first outer radial clearance R1 between impeller 118 and pump housing 117 to vary as impeller 118 imparts the energy to the fluid.

The translation of eye axis AE around a post axis AP as impeller 118 imparts energy to the fluid may generate a pulsating flow of the fluid. The pulsating flow may define a substantially continuous pressure waveform which substantially oscillates between the maximum pressure and the minimum pressure.

Control circuitry 120 may be configured to alter the operation of pump 104 to control a pressure range exhibited by the pulsating flow as impeller 118 imparts the energy to the fluid. Control circuitry 120 may alter the operation of pump 104 (e.g., a rotational speed of impeller 118) to control a wave period of the pulsating flow as impeller 118 imparts energy to the fluid. Stator 158, 160 may magnetically interact with impeller 118 to cause impeller 118 to rotate around eye axis AE. In examples, the stator and/or the impeller define a motor magnetic field to cause the impeller to rotate around the eye axis. Control circuitry 120 may control a rotational speed and/or strength of the motor magnetic field to control a rotational speed of the impeller.

In the example shown in FIG. 7, control circuitry 120 modifies a bearing magnetic field of magnetic bearing 156 to modify an operation of MCS device 102 (704). In examples, control circuitry 120 may increase a current and/or voltage supplied to windings 170, 172 to modify the operation of MCS device 102 (e.g., pump 104). Control circuitry 120 may alter the bearing magnetic field to increase or decrease magnetic forces between impeller 118 and post 146 to modify the operation of MCS device 102. For example, control circuitry may alter the bearing magnetic field to increase a first magnetic force acting in a first direction between impeller 118 and post 146 and decrease a second magnetic force acting in a second direction opposite the first direction to modify the operation of MCS device 102. Control circuitry may modify the bearing magnetic field to modify the radial distribution (e.g., with respect to post axis AP) of the magnetic forces acting between impeller 118 and post 146. In examples, control circuitry modifies the bearing magnetic field to increase and/or decrease a maximum outer radial clearance, minimum outer radial clearance, maximum inner radial clearance, and/or minimum inner radial clearance. Control circuitry 120 may modify the magnetic field strength of the bearing magnetic field to modify the operation of MCS device 102.

Control circuitry 120 may receive a flow signal from sensor 121 and modify the bearing magnetic field and/or motor magnetic field based on the flow signal. Sensor 121 may generate a flow signal indicative of the pulsating flow and/or the characteristic of MCS device 102 indicating the pulsating flow, such as a flow rate of the blood flow, a pressure of the blood flow, a position of impeller 118, an electrical characteristic of MCS 102 (e.g., pump 104), and/or a mechanical wave generated by MCS device 102. Control circuitry 120 may analyze evaluate the flow signal to indicate MCS device 102 is producing a pulsating flow (e.g., based on the variance and/or oscillation of a wave form exhibited by the flow signal). In examples, control circuitry 120 may evaluate the pulsating flow generated by MCS device 102 by evaluating a plurality of frequency peaks, periods, or other characteristics of the wave form of the flow signal.

Control circuitry 120 may monitor MCS device 102 to evaluate, assess, determine, analyze, monitor, and or otherwise confirm that pump 104 is operating in a condition causing eye axis AE to translate around post axis AP as impeller 118 rotates around eye axis AE. Control circuitry 120 may evaluate, assess, analyze, and or otherwise monitor the flow signal to determine if MCS device 102 is producing a pulsating flow. Control circuitry 120 may alter an operating characteristic of pump 104 to cause the pulsating flow based on the flow signal.

Control circuitry 120 may control an operation of MCS device 102 based on a sensed physiological parameter of the patient 103. Monitor 128 may detect a physiological parameter of patient 103 (e.g., a cardiac signal such as an ECG, ECM, an activity level of patient 103, a mechanical wave of heart 101, an electrical field of heart 101, or another physiological parameter) and communicate a signal indicative of the physiological parameter to computing device 130 (e.g., via communication link 134) and/or control circuitry 120. Control circuitry 120 may alter an operation of pump 104 based on the indicative signal from monitor 128 and/or a communication from computing device 130.

The techniques described in this disclosure, including those attributed to system 100, control circuitry 120, computing device 130, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry. The term “processor,” “processing circuitry,” “controller,” or “control circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

This disclosure includes the following non-limiting examples.

Example 1: A mechanical circulatory support device comprising: a housing including a fluid inlet and a fluid outlet, wherein the housing defines a volume fluidically coupling the fluid inlet and the fluid outlet; a post mechanically supported by the housing within the volume, wherein the post defines a post axis, and wherein the post mechanically supports an inner ring; and an impeller mechanically supporting an outer ring, wherein the impeller defines an eye surrounding the post and an eye axis extending through the eye, wherein the inner ring is configured to magnetically interact with the outer ring to establish a radial clearance between the inner ring and the outer ring, and wherein the inner ring is configured to magnetically interact with the outer ring to cause the eye axis to translate around the post axis as the impeller rotates around the eye axis when the impeller imparts energy to a fluid flowing from the fluid inlet to the fluid outlet.

Example 2: The mechanical circulatory support device of example 1, wherein the eye axis and the post axis are substantially parallel.

Example 3: The mechanical circulatory support device of example 1 or example 2, wherein the impeller defines an outer radial clearance between an outer perimeter of the impeller and the housing, and wherein the inner ring is configured to magnetically interact with the outer ring to cause the outer radial clearance to vary as the impeller rotates around the eye axis.

Example 4: The mechanical circulatory support device of any of examples 1-3, wherein the impeller is configured to cause a pulsating flow of the fluid at the fluid outlet when the impeller imparts the velocity to the fluid.

Example 5: The mechanical circulatory support device of any of examples 1-4, wherein the inner ring and the outer ring are configured to define a magnetic field to cause the inner ring to magnetically interact with the outer ring, and wherein at least one of the inner ring or the outer ring is configured to cause the magnetic field to cause magnetic forces between the inner ring and the outer ring that vary around a perimeter defined by the inner ring.

Example 6: The mechanical circulatory support device of example 5, wherein at least one of the inner ring or the outer ring includes a permanent magnet, and wherein the permanent magnet defines a structural feature configured to cause the magnetic forces to vary around the perimeter defined by the inner ring.

Example 7: The mechanical circulatory support device of any of examples 1-6 wherein at least one of the inner ring or the outer ring includes a winding configured to generate an electromagnetic field configured to cause the inner ring to magnetically interact with the outer ring, wherein the control circuitry is configured to control a magnetic strength of the electromagnetic field using the winding.

Example 8: The mechanical circulatory support device of any of examples 1-7, wherein the impeller includes a pole piece, the pump further comprising a stator configured to generate a stator magnetic field, wherein the stator is configured to cause the stator magnetic field to magnetically interact with the pole piece to cause the impeller to rotate around the eye axis.

Example 9: The mechanical circulatory support device of any of examples 1-8, further comprising control circuitry, wherein the stator includes a stator winding configured to generate a rotating stator magnetic field, and wherein the control circuitry is configured to control a rotational speed of the rotating stator magnetic field using the stator winding.

Example 10: The mechanical circulatory support device of any of examples 1-8, wherein the inner ring is integrally formed with the post, the outer ring is integrally formed with the impeller, or the inner ring is integrally formed with the post and the outer ring is integrally formed with the impeller.

Example 11: The mechanical circulatory support device of any of examples 1-10, wherein the housing is configured to cause the fluid to flow through the radial clearance between the inner ring and the outer ring when the fluid flows from the fluid inlet to the fluid outlet.

Example 12: The mechanical circulatory support device of any of examples 1-11, further comprising: a sensor configured to provide a flow signal indicative of a flow of the fluid; and control circuitry configured to determine when the eye axis translates around the post axis based on the flow signal.

Example 13: The mechanical circulatory support device of any of examples 1-12, further comprising: an inflow line configured to receive a blood flow from a heart of a patient and provide the blood flow to the fluid inlet; and an outflow line configured to receive the blood flow from the fluid outlet and provide the blood flow to vasculature of the patient.

Example 14: The mechanical circulatory support device of example 13, further comprising control circuitry configured to: receive one or more signals indicative of a physiological parameter of the heart of the patient; and control at least one of the radial clearance or a rotational speed of the impeller based on the one or more signals.

Example 15: The mechanical circulatory support device of example 14, further comprising a display, wherein the control circuitry is configured to output, via the display, an indication of at least one of the physiological parameters of the heart, an operating condition of the pump, or a performance characteristic of the pump.

Example 16: A heart pump, comprising: a housing including a fluid inlet and a fluid outlet, wherein the housing defines a volume fluidically coupling the fluid inlet and the fluid outlet; a stator configured to generate a stator magnetic field; a post mechanically supported by the housing within the volume, wherein the post mechanically supports an inner ring; and an impeller configured to impart energy to a fluid flowing from the fluid inlet to the fluid outlet, wherein the impeller defines an outer radial clearance between an outer perimeter of the impeller and the housing, and wherein the inner ring is configured to magnetically interact with an outer ring mechanically supported by the impeller to cause the outer radial clearance to vary.

Example 17: The heart pump of example 16, wherein the post defines a post axis, and wherein the impeller defines an eye surrounding the post and an eye axis extending through the eye, and wherein the inner ring is configured to magnetically interact with the outer ring to cause the eye axis to translate around the post axis as the impeller rotates around the eye axis to impart velocity to the fluid.

Example 18: The heart pump of example 16 or example 17, wherein the inner ring is configured to magnetically interact with the outer ring to establish a radial clearance between the inner ring and the outer ring.

Example 19: A method, comprising: controlling, by control circuitry, an impeller of a medical pump to rotate within a housing, the medical pump comprising: a housing including a fluid inlet and a fluid outlet, wherein the housing defines a volume fluidically coupling the fluid inlet and the fluid outlet; a post mechanically supported by the housing within the volume, wherein the post defines a post axis, and wherein the post mechanically supports an inner ring; and the impeller mechanically supporting an outer ring, wherein the impeller defines an eye surrounding the post and an eye axis extending through the eye, wherein the inner ring is configured to magnetically interact with the outer ring to establish a radial clearance between the inner ring and the outer ring, and wherein the inner ring is configured to magnetically interact with the outer ring to cause the eye axis to translate around the post axis as the impeller rotates around the eye axis when the impeller imparts energy to a fluid flowing from the fluid inlet to the fluid outlet; and modifying, by the control circuitry, a magnetic field causing the magnetic interaction between the inner ring and the outer ring.

Example 20: The method of example 19, further comprising receiving, by the control circuitry, at least one of a flow signal indicative of a pressure of the fluid as the impeller imparts the velocity to the fluid or a physiological signal indicative of a physiological parameter of a patient, wherein modifying the magnetic field comprises modifying the magnetic field based on the flow signal or the physiological signal

Various examples of the disclosure have been described. Any combination of the described systems, operations, or functions is contemplated. These and other examples are within the scope of the following claims.

What is claimed is:

1. A mechanical circulatory support device comprising:
a housing including a fluid inlet and a fluid outlet, wherein the housing defines a volume fluidically coupling the fluid inlet and the fluid outlet;
a post mechanically supported by the housing within the volume, wherein the post defines a post axis, and wherein the post mechanically supports an inner ring; and
an impeller mechanically supporting an outer ring,
wherein the impeller defines an eye surrounding the post and an eye axis extending through the eye,
wherein the inner ring is configured to magnetically interact with the outer ring to establish a radial clearance between the inner ring and the outer ring, and
wherein the inner ring is configured to magnetically interact with the outer ring to cause the eye axis to translate around the post axis as the impeller rotates around the eye axis when the impeller imparts energy to a fluid flowing from the fluid inlet to the fluid outlet.

2. The mechanical circulatory support device of claim 1, wherein the eye axis and the post axis are substantially parallel.

3. The mechanical circulatory support device of claim 1, wherein the impeller defines an outer radial clearance between an outer perimeter of the impeller and the housing, and wherein the inner ring is configured to magnetically interact with the outer ring to cause the outer radial clearance to vary as the impeller rotates around the eye axis.

4. The mechanical circulatory support device of claim 1, wherein the impeller is configured to cause a pulsating flow of the fluid at the fluid outlet when the impeller imparts the velocity to the fluid.

5. The mechanical circulatory support device of claim 1, wherein the inner ring and the outer ring are configured to define a magnetic field to cause the inner ring to magnetically interact with the outer ring, and wherein at least one of the inner ring or the outer ring is configured to cause the magnetic field to cause magnetic forces between the inner ring and the outer ring that vary around a perimeter defined by the inner ring.

6. The mechanical circulatory support device of claim 5, wherein at least one of the inner ring or the outer ring includes a permanent magnet, and wherein the permanent magnet defines a structural feature configured to cause the magnetic forces to vary around the perimeter defined by the inner ring.

7. The mechanical circulatory support device of claim 1, further comprising control circuitry, wherein at least one of the inner ring or the outer ring includes a winding configured to generate an electromagnetic field configured to cause the inner ring to magnetically interact with the outer ring, wherein the control circuitry is configured to control a magnetic strength of the electromagnetic field using the winding.

8. The mechanical circulatory support device of claim 1, wherein the impeller includes a pole piece, the pump further comprising a stator configured to generate a stator magnetic field, wherein the stator is configured to cause the stator magnetic field to magnetically interact with the pole piece to cause the impeller to rotate around the eye axis.

9. The mechanical circulatory support device of claim 8, further comprising control circuitry, wherein the stator includes a stator winding configured to generate a rotating stator magnetic field, and wherein the control circuitry is configured to control a rotational speed of the rotating stator magnetic field using the stator winding.

10. The mechanical circulatory support device of claim 1, wherein the inner ring is integrally formed with the post, the outer ring is integrally formed with the impeller, or the inner ring is integrally formed with the post and the outer ring is integrally formed with the impeller.

11. The mechanical circulatory support device of claim 1, wherein the housing is configured to cause the fluid to flow through the radial clearance between the inner ring and the outer ring when the fluid flows from the fluid inlet to the fluid outlet.

12. The mechanical circulatory support device of claim 1, further comprising:
a sensor configured to provide a flow signal indicative of a flow of the fluid; and
control circuitry configured to determine when the eye axis translates around the post axis based on the flow signal.

13. The mechanical circulatory support device of claim 1, further comprising:
an inflow line configured to receive a blood flow from a heart of a patient and provide the blood flow to the fluid inlet; and
an outflow line configured to receive the blood flow from the fluid outlet and provide the blood flow to vasculature of the patient.

14. The mechanical circulatory support device of claim 13, further comprising control circuitry configured to:
receive one or more signals indicative of a physiological parameter of the heart of the patient; and
control at least one of the radial clearance or a rotational speed of the impeller based on the one or more signals.

15. The mechanical circulatory support device of claim 14, further comprising a display, wherein the control circuitry is configured to output, via the display, an indication of at least one of the physiological parameters of the heart, an operating condition of the pump, or a performance characteristic of the pump.

16. A heart pump, comprising:
a housing including a fluid inlet and a fluid outlet, wherein the housing defines a volume fluidically coupling the fluid inlet and the fluid outlet;
a stator configured to generate a stator magnetic field;
a post mechanically supported by the housing within the volume, wherein the post mechanically supports an inner ring; and
an impeller configured to impart energy to a fluid flowing from the fluid inlet to the fluid outlet,
wherein the impeller defines an outer radial clearance between an outer perimeter of the impeller and the housing, and
wherein the inner ring is configured to magnetically interact with an outer ring mechanically supported by the impeller to cause the outer radial clearance to vary.

17. The heart pump of claim 16, wherein the post defines a post axis, and wherein the impeller defines an eye surrounding the post and an eye axis extending through the eye, and wherein the inner ring is configured to magnetically interact with the outer ring to cause the eye axis to translate around the post axis as the impeller rotates around the eye axis to impart velocity to the fluid.

18. The heart pump of claim 16, wherein the inner ring is configured to magnetically interact with the outer ring to establish a radial clearance between the inner ring and the outer ring.

19. A method, comprising:
controlling, by control circuitry, an impeller of a medical pump to rotate within a housing, the medical pump comprising:
a housing including a fluid inlet and a fluid outlet, wherein the housing defines a volume fluidically coupling the fluid inlet and the fluid outlet;
a post mechanically supported by the housing within the volume, wherein the post defines a post axis, and wherein the post mechanically supports an inner ring; and
the impeller mechanically supporting an outer ring, wherein the impeller defines an eye surrounding the post and an eye axis extending through the eye,
wherein the inner ring is configured to magnetically interact with the outer ring to establish a radial clearance between the inner ring and the outer ring, and
wherein the inner ring is configured to magnetically interact with the outer ring to cause the eye axis to translate around the post axis as the impeller rotates around the eye axis when the impeller imparts energy to a fluid flowing from the fluid inlet to the fluid outlet; and
modifying, by the control circuitry, a magnetic field causing the magnetic interaction between the inner ring and the outer ring.

20. The method of claim 19, further comprising receiving, by the control circuitry, at least one of a flow signal indicative of a pressure of the fluid as the impeller imparts the velocity to the fluid or a physiological signal indicative of a physiological parameter of a patient, wherein modifying the magnetic field comprises modifying the magnetic field based on the flow signal or the physiological signal.
